SEQUENCE LISTING (86 sequences)

SEQ ID NO: 1 - protein, 29 aa, Homo sapiens
ERLRRRIELCRRHHSTCEARYEAVSPERL

SEQ ID NO: 2 - protein, 44 aa, Homo sapiens
VMERLRRRIELCRRHHSTCEARYEAVSPERLELERQHTFALHQR

SEQ ID NO: 3 - protein, 44 aa, Homo sapiens
IVERLRARIAVCRQHHLSCEGRYERGRAESSDRERESTLQLLSL

SEQ ID NO: 4 - protein, 44 aa, Homo sapiens
VVERLRQRIEGCRRHHVNCENRYQQAQVEQLELERRDTVSLYQR

SEQ ID NO: 5 - protein, 59 aa, Homo sapiens
HSAVMERLRRRIELCRRHHSTCEARYEAVSPERLELERQHTFALHQRCIQAKAKRAGKH

SEQ ID NO: 6 - protein, 60 aa, Homo sapiens
HSAIVERLRARIAVCRQHHLSCEGRYERGRAESSDRERESTLQLLSLVQHGQGARKAGKH

SEQ ID NO: 7 - protein, 60 aa, Homo sapiens
AVPKHSTVVERLRQRIEGCRRHHVNCENRYQQAQVEQLELERRDTVSLYQRTLEQRAKKS

SEQ ID NO: 8 - protein, 16 aa, Homo sapiens
ERLRRRIELCRRHHST

SEQ ID NO: 9 - protein, 17 aa, Homo sapiens
ERLRARIAVCRQHHLSC

SEQ ID NO: 10 - protein, 16 aa, Homo sapiens
ERLRQRIEGCRRHHVN

SEQ ID NO: 11 - protein, 29 aa, Homo sapiens
ERLRARIAVCRQHHLSCEGRYERGRAESS

SEQ ID NO: 12 - protein, 16 aa, Artificial Sequence
  Description of Artificial Sequence Synthetic peptide
ERXXRRXXXXRXHHSX

SEQ ID NO: 13 - protein, 21 aa, Artificial Sequence
  Description of Artificial Sequence Synthetic peptide
ERXXRRXXXXRXHHSXXXARX

SEQ ID NO: 14 - protein, 29 aa, Artificial Sequence
  Description of Artificial Sequence Synthetic peptide
ERXXRRXXXXRXHHSXXXARXEAVSPERL

SEQ ID NO: 15 - protein, 29 aa, Artificial Sequence
  Description of Artificial Sequence Synthetic peptide
ERXXARXXXXRXHHLXXXGRXERGRAESS

SEQ ID NO: 16 - protein, 21 aa, Artificial Sequence
  Description of Artificial Sequence Synthetic peptide
ERXXARXXXXRXHHLXXXGRX

SEQ ID NO: 17 - protein, 29 aa, Artificial Sequence
  Description of Artificial Sequence Synthetic peptide
ERXXARXXXXRXHHLXXXGRXERGRAESS

SEQ ID NO: 18 - protein, 29 aa, Artificial Sequence
  Description of Artificial Sequence Synthetic peptide
ERXXQRXXXXRXHHVXXXNRXQQAQVEQL

SEQ ID NO: 19 - protein, 21 aa, Artificial Sequence
  Description of Artificial Sequence Synthetic peptide
ERXXQRXXXXRXHHVXXXNRX

SEQ ID NO: 20 - protein, 29 aa, Artificial Sequence
  Description of Artificial Sequence Synthetic peptide
ERXXQRXXXXRXHHVXXXNRXQQAQVEQL

SEQ ID NO: 21 - protein, 29 aa, Homo sapiens
ERLRQRIEGCRRHHVNCENRYQQAQVEQL

SEQ ID NO: 22 - protein, 6 aa, Artificial Sequence
  Description of Artificial Sequence Synthetic 6xHis tag
HHHHHH

SEQ ID NO: 23 - protein, 55 aa, Artificial Sequence
  Description of Artificial Sequence Synthetic polypeptide
HSAVMERLRRRIELCRRHHSTCEARYEAVSPERLELERQHTFALHQRCIQAKAKR

SEQ ID NO: 24 - protein, 22 aa, Homo sapiens
MERLRRRIELCRRHHSTCEARY

SEQ ID NO: 25 - protein, 22 aa, Homo sapiens
VERLRARIAVCRQHHLSCEGRY

SEQ ID NO: 26 - protein, 22 aa, Homo sapiens
VERLRQRIEGCRRHHVNCENRY

SEQ ID NO: 27 - protein, 22 aa, Mus musculus
MERLRRRIELCRRHHSTCEARY

SEQ ID NO: 28 - protein, 22 aa, Xenopus laevis
MERLRRRIELCRRHHGSCESRY

SEQ ID NO: 29 - protein, 22 aa, Drosophila melanogaster
VDRLRRRMENYRRRQTDCVPRY

SEQ ID NO: 30 - protein, 22 aa, Caenorhabditis elegans
LNAFHSGEELHRQRSELARANY

SEQ ID NO: 31 - protein, 22 aa, Danio rerio
MERLRRRIELFRRHHTGCENRY

SEQ ID NO: 32 - protein, 56 aa, Artificial Sequence
  Description of Artificial Sequence Synthetic polypeptide
HSAVLERLRRRIELCRRHHSTCEARYEAVSPERLELERQHTFALHQRCIQAKAKRA

SEQ ID NO: 33 - protein, 18 aa, Artificial Sequence
  Description of Artificial Sequence Synthetic peptide
WAERLRRRIXLCRXHHST

SEQ ID NO: 34 - protein, 18 aa, Artificial Sequence
  Description of Artificial Sequence Synthetic peptide
WAERLRRRIXLVRXHHST

SEQ ID NO: 35 - protein, 18 aa, Artificial Sequence
  Description of Artificial Sequence Synthetic peptide
WAERLRRRIXLLRXHHST

SEQ ID NO: 36 - protein, 18 aa, Artificial Sequence
  Description of Artificial Sequence Synthetic peptide
WAERLRRRIXLFRXHHST

SEQ ID NO: 37 - protein, 18 aa, Artificial Sequence
  Description of Artificial Sequence Synthetic peptide
WAERLRRRIXICRXHHST

SEQ ID NO: 38 - protein, 18 aa, Artificial Sequence
  Description of Artificial Sequence Synthetic peptide
WAERLRRRIXFCRXHHST

SEQ ID NO: 39 - protein, 18 aa, Artificial Sequence
  Description of Artificial Sequence Synthetic peptide
WAERLRRRIXWCRXHHST

SEQ ID NO: 40 - protein, 18 aa, Artificial Sequence
  Description of Artificial Sequence Synthetic peptide
WAERLRRRLXLCRXHHST

SEQ ID NO: 41 - protein, 18 aa, Artificial Sequence
  Description of Artificial Sequence Synthetic peptide
WAERLRRRLXLCRXHHST

SEQ ID NO: 42 - protein, 18 aa, Artificial Sequence
  Description of Artificial Sequence Synthetic peptide
WAERLRRRFXLCRXHHST

SEQ ID NO: 43 - protein, 18 aa, Artificial Sequence
  Description of Artificial Sequence Synthetic peptide
WAERLRRRWXLCRXHHST

SEQ ID NO: 44 - protein, 18 aa, Artificial Sequence
  Description of Artificial Sequence Synthetic peptide
WAERLRRRIXLCRXHHST

SEQ ID NO: 45 - protein, 18 aa, Artificial Sequence
  Description of Artificial Sequence Synthetic peptide
WAERFRRRIXLCRXHHST

SEQ ID NO: 46 - protein, 18 aa, Artificial Sequence
  Description of Artificial Sequence Synthetic peptide
WAERWRRRIXLCRXHHST

SEQ ID NO: 47 - protein, 18 aa, Artificial Sequence
  Description of Artificial Sequence Synthetic peptide
WAERLRRRIXYCRXHHST

SEQ ID NO: 48 - protein, 18 aa, Artificial Sequence
  Description of Artificial Sequence Synthetic peptide
WAERLRRRIELCRRHHST

SEQ ID NO: 49 - protein, 18 aa, Artificial Sequence
  Description of Artificial Sequence Synthetic peptide
WARELRREIXLCRXHHST

SEQ ID NO: 50 - protein, 29 aa, Artificial Sequence
  Description of Artificial Sequence Synthetic polypeptide
ERLRRRIXLCRXHHSTCEARYEAVSPERL

SEQ ID NO: 51 - protein, 18 aa, Artificial Sequence
  Description of Artificial Sequence Synthetic peptide
WAERLRRRIXLCRXHHST

SEQ ID NO: 52 - protein, 18 aa, Artificial Sequence
  Description of Artificial Sequence Synthetic peptide
WAERWRRRIXLFRXHHST

SEQ ID NO: 53 - protein, 18 aa, Artificial Sequence
  Description of Artificial Sequence Synthetic peptide
WAERWRRRIXFCRXHHST

SEQ ID NO: 54 - protein, 18 aa, Artificial Sequence
  Description of Artificial Sequence Synthetic peptide
WAERWRRRIXWCRXHHST

SEQ ID NO: 55 - protein, 18 aa, Artificial Sequence
  Description of Artificial Sequence Synthetic peptide
WAERWRRRLXLFRXHHST

SEQ ID NO: 56 - protein, 18 aa, Artificial Sequence
  Description of Artificial Sequence Synthetic peptide
WAERWRRRLXWCRXHHST

SEQ ID NO: 57 - protein, 18 aa, Artificial Sequence
  Description of Artificial Sequence Synthetic peptide
WAERWRRRLXWVRXHHST

SEQ ID NO: 58 - protein, 18 aa, Artificial Sequence
  Description of Artificial Sequence Synthetic peptide
WAERWRRRLXWFRXHHST

SEQ ID NO: 59 - protein, 18 aa, Artificial Sequence
  Description of Artificial Sequence Synthetic peptide
WAERWRRRLXYCRXHHST

SEQ ID NO: 60 - protein, 18 aa, Artificial Sequence
  Description of Artificial Sequence Synthetic peptide
WAERWRRRLXYVRXHHST

SEQ ID NO: 61 - protein, 18 aa, Artificial Sequence
  Description of Artificial Sequence Synthetic peptide
WAERKRRRIXLCRXHHST

SEQ ID NO: 62 - protein, 18 aa, Artificial Sequence
  Description of Artificial Sequence Synthetic peptide
WAERWRRRIXYCRXHHST

SEQ ID NO: 63 - protein, 18 aa, Artificial Sequence
  Description of Artificial Sequence Synthetic peptide
WAERWRRRIXYFRXHHST

SEQ ID NO: 64 - protein, 15 aa, Artificial Sequence
  Description of Artificial Sequence Synthetic peptide
WAELERXHTFXLHQR

SEQ ID NO: 65 - protein, 18 aa, Artificial Sequence
  Description of Artificial Sequence Synthetic peptide
WARELRREIXLCRXHHST

SEQ ID NO: 66 - protein, 23 aa, Artificial Sequence
  Description of Artificial Sequence Synthetic peptide
WAERLRRRIXLCRXHHSTCEARY

SEQ ID NO: 67 - protein, 23 aa, Artificial Sequence
  Description of Artificial Sequence Synthetic peptide
WAERWRRRIXLFRXHHSTCEARY

SEQ ID NO: 68 - protein, 23 aa, Artificial Sequence
  Description of Artificial Sequence Synthetic peptide
WAERWRRRIXFCRXHHSTCEARY

SEQ ID NO: 69 - protein, 23 aa, Artificial Sequence
  Description of Artificial Sequence Synthetic peptide
WAERWRRRIXWCRXHHSTCEARY

SEQ ID NO: 70 - protein, 23 aa, Artificial Sequence
  Description of Artificial Sequence Synthetic peptide
WAERLRRRIXLCRXHHSTFEARY

SEQ ID NO: 71 - protein, 23 aa, Artificial Sequence
  Description of Artificial Sequence Synthetic peptide
WAERLRRRIXLCRXHHSXCEARY

SEQ ID NO: 72 - protein, 23 aa, Artificial Sequence
  Description of Artificial Sequence Synthetic peptide
WAERLXRRIXLCRXHHSTCEARY

SEQ ID NO: 73 - protein, 23 aa, Artificial Sequence
  Description of Artificial Sequence Synthetic peptide
WAERWRRRLXYCRXHHSTCEARY

SEQ ID NO: 74 - protein, 31 aa, Artificial Sequence
  Description of Artificial Sequence Synthetic polypeptide
WAERWRRRLXYCRXHHSTCEARYEAVSPERL

SEQ ID NO: 75 - protein, 31 aa, Artificial Sequence
  Description of Artificial Sequence Synthetic polypeptide
WAERWRRRLXYVRXHHSTCEARYEAVSPERL

SEQ ID NO: 76 - protein, 31 aa, Artificial Sequence
  Description of Artificial Sequence Synthetic polypeptide
WAERWRRRLXWCRXHHSTCEARYEAVSPERL

SEQ ID NO: 77 - protein, 23 aa, Artificial Sequence
  Description of Artificial Sequence Synthetic peptide
WAERWRRRLXLFRXHHSTFEARY

SEQ ID NO: 78 - protein, 23 aa, Artificial Sequence
  Description of Artificial Sequence Synthetic peptide
WAERWRRRLXWCRXHHSTFEARY

SEQ ID NO: 79 - protein, 23 aa, Artificial Sequence
  Description of Artificial Sequence Synthetic peptide
WAERWRRRLXLCRXHHSTCEARX

SEQ ID NO: 80 - protein, 23 aa, Artificial Sequence
  Description of Artificial Sequence Synthetic peptide
WAERWRRRLXLCRXHHSTCEARA

SEQ ID NO: 81 - protein, 23 aa, Artificial Sequence
  Description of Artificial Sequence Synthetic peptide
WAERWRRRLXLCRXHHSTCEARA

SEQ ID NO: 82 - protein, 23 aa, Artificial Sequence
  Description of Artificial Sequence Synthetic peptide
WAERWRRRLXLCRXHHSTXEARY

SEQ ID NO: 83 - protein, 18 aa, Artificial Sequence
  Description of Artificial Sequence Synthetic peptide
WAERWRRRIXLMRXHHST

SEQ ID NO: 84 - protein, 23 aa, Artificial Sequence
  Description of Artificial Sequence Synthetic peptide
WAERWRRRLXYFRXHHSTFEARY

SEQ ID NO: 85 - protein, 23 aa, Artificial Sequence
  Description of Artificial Sequence Synthetic peptide
WAERWRRRLXYFRXHHSTCEARA

SEQ ID NO: 86 - protein, 23 aa, Artificial Sequence
  Description of Artificial Sequence Synthetic peptide
WAERWRRRLXYFRXHHSTFEARA

(19) United States
(12) Patent Application Publication
(10) Pub. No.: US 2014/0256912 A1
(43) Pub. Date: Sep. 11, 2014

(54) Stabilized Variant MAML Peptides and Uses Thereof

(71) Applicants: Raymond Earle Moellering, Cambridge, MA (US); Gregory L. Verdine, Newton, MA (US)

(72) Inventors: Raymond Earle Moellering, Cambridge, MA (US); Gregory L. Verdine, Newton, MA (US)

(73) Assignee: PRESIDENT AND FELLOWS OF HARVARD COLLEGE, Cambridge (US)

(21) Appl. No.: 14/127,039

(22) PCT Filed: Jun. 15, 2012
(86) PCT No.: PCT/US2012/042719

§ 371 Date: Mar. 19, 2014

Related U.S. Application Data
(60) Provisional application No. 61/498,477, filed on Jun. 17, 2011.

(51) Int. Cl. C07K 14/47; C07K 7/08

(52) U.S. Cl. CPC C07K 14/47; C07K 7/08
USPC 530/324; 530/326

(57) ABSTRACT

Internally cross-linked peptides derived from human MAML and derivatives thereof which exhibit affinity for the ICN1-CSL complex are described and characterized. The peptides can interfere with NOTCH signaling and are thus useful for treating various disorders, including certain cancers.

### BACKGROUND

Aberrant transcription factor function is a hallmark of tumor development and progression. Deregulation of these critical regulatory molecules can result from numerous genetic events including mutation, translocation or amplification of upstream regulatory proteins such as kinases (e.g. BCR-Abl, b-Raf and k-Ras), deletion or inactivating mutation of protein phosphatases (e.g. PTEN), altered growth factor-receptor signaling (e.g. VEGF-VEGFR) or direct mutation, deletion, amplification or fusion of transcription factors themselves (e.g. MYC, p53 and NOTCH1). In each of these cases, altered signaling cascades ultimately lead to differential activity of one or more transcription factors and the induction of abnormal gene expression networks¹. While many “driver” oncogenes have been characterized, it is ultimately these networks that contribute to the malignant phenotype and cancer progression. Despite their critical role in genetic diseases such as cancer however, transcription factors have proven to be extremely challenging targets for the development of traditional small molecule drugs.

The Notch signaling pathway is a prototypical example of an oncogenic transcriptional network driven by overactive signaling through the multi-protein NOTCH transactivation complex. Normal Notch signaling is integral to a variety of developmental processes, including neural precursor specification, hematopoietic stem cell maintenance and lineage determination^{2,3}. The tight regulation of these processes derives in large part from the exquisite control ordinarily imposed by the cell over the duration and dosage of signals emanating from the activated Notch pathway. Aberrations in Notch pathway function and control are linked with a wide variety of disorders in humans. Mutations that disrupt NOTCH protein function have been observed in numerous developmental disorders, including CADASIL⁴, congenital aortic valve defects⁵ and Allagille syndrome⁶. On the other hand, genetic alterations that cause inappropriate, sustained activation of the Notch pathway are causally linked with cancer. Indeed, human NOTCH1 was discovered on the basis of its involvement in a t(7;9) chromosomal translocation observed in patients with T-cell acute lymphoblastic leukemia (T-ALL)⁷. Subsequently, various activating mutations in NOTCH1 have been discovered in greater than 50% of patients with T-ALL⁸. Following these seminal discoveries in T-ALL, additional genetic insults that potentiate Notch signaling have been identified in many other forms of cancer including those of the breast⁹, ovaries¹⁰, lungs^{11,12}, pancreas¹³ and gastrointestinal tract as well as in melanoma¹⁴, multiple myeloma¹⁵ and medulloblastoma. Additionally, aberrant Notch signaling has recently been implicated in the pathogenesis of numerous chronic diseases beyond cancer, including inflammatory atherosclerosis¹⁶, glomerulosclerosis¹⁷, osteoporosis¹⁸ and arterial hypertension

Given the extensive causal relationships between NOTCH proteins and disease, considerable interest exists in the development of pharmacologic agents that antagonize the Notch pathway. Following receptor activation, NOTCH proteins undergo two sequential proteolytic cleavage events by an ADAM family metalloprotease²⁰ and the γ-secretase complex²¹⁻²³, respectively. Intramembrane cleavage of NOTCH receptors by γ-secretase releases an intracellular domain of NOTCH (ICN), which translocates to the nucleus and forms the active NOTCH transcriptional complex (NTC) with the transcription factor CSL and co-activators of the Mastermind-like family (MAML1-3 in humans) (FIG. 1a)^{24-27,28}. Several classes of therapeutics have been developed to inhibit NOTCH ligands^{29,30}, the extracellular domains of NOTCH receptors^{31,32} and the γ-secretase complex³³⁻³⁶.

WO 2008/061192 describes certain cross-linked peptides derived from MAML1 that were tested these for aqueous solubility, strength of binding to the ICN-CSL complex, and for efficient of cellular penetration. One such peptide, SAHM1 was found to specifically bind the ICN1-CSL complex and competitively inhibit binding of recombinant dnMAML1 as well as full-length MAML1. When incubated with human T-ALL cells, SAHM1 was shown to inhibit the expression of a panel of canonical Notch target genes (HES1, MYC, DTX1). A more comprehensive investigation employing gene expression profiling and gene set enrichment analysis demonstrated that SAHM1 produces a transcriptional signature of Notch gene repression in human and murine T-ALL cells—indeed one that showed striking correspondence to that produced by treatment with a small-molecule γ-secretase inhibitor (GSI). Direct blockade of NOTCH-CSL transcriptional activation was found to induce NOTCH-specific anti-proliferative effects in human T-ALL cell lines as well as in a bioluminescent murine model of T-ALL driven by a clinically observed mutant NOTCH1 allele.

### SUMMARY

Described below are stably cross-linked peptides related to a portion of human MAML1 (“stapled MAML1 peptides”). These cross-linked peptides contain at least two modified amino acids that together form an internal (intramolecular) cross-link between the alpha carbons of the two modified amino acids that can help to stabilize the alpha-helical secondary structure of the peptide (see U.S. Pat. No. 7,192,173 and Verdine et al. 2012 Methods in Enzymology 503:3) In some cases the peptide includes four (6, 8 or 10) modified amino acids, pairs of which form an internal cross-link. Such peptides have two (3, 4 or 5) internal cross-links separated by one or more, e.g., three amino acids. In some cases the peptide contains three modified amino acids, the middle one of which forms a cross-link (between alpha carbons) with each of the two flanking amino acids. Such cross-linked peptides, which also have two internal cross-links, are sometimes referred to as “stitched” peptides and are described in US 2010/0184645.

A cross-linked polypeptide described herein can have improved biological activity relative to a corresponding polypeptide that is not cross-linked. The cross-linked MAML1 peptides can bind to the ICN1-CSL complex and competitively inhibit binding of recombinant MAML1 or full-length MAML proteins (MAML1-3) to ICN1-CSL complexes. Certain active peptides are expected to inhibit the expression of one or more Notch-regulated genes (HES1, MYC, DTX1 and others) in T-ALL cells or other cells in which Notch signaling is active, an expectation that is supported by Notch 1-dependent reporter gene studies. The internally cross-linked MAML peptides described herein can be used therapeutically, e.g., to treat a variety of cancers or Notch-dependent diseases in a subject, for example, cancers and other disorders characterized by undesirable activation of a Notch receptors or Notch-activated gene(s).

The cross-linked MAML1 peptides described herein are variants of a portion of human MAML1 and could include amino acid substitutions from other MAML isoforms (MAML2 and MAML3) or novel amino acid mutations. The sequence of a relevant portion of human MAML1 (starts at amino acid 21 of MAML1) is depicted below:

(SEQ ID NO: 1):
Glu₁Arg₂Leu₃Arg₄Arg₅Arg₆Ile₇Glu₈Leu₉Cys₁₀Arg₁₁Arg₁₂
His₁₃His₁₄Ser₁₅Thr₁₆Cys₁₇Glu₁₈Ala₁₉Arg₂₀Tyr₂₁Glu₂₂
Ala₂₃Val₂₄Ser₂₅Pro₂₆Glu₂₇Arg₂₈Leu₂₉ (SEQ ID NO: 1)

Other relevant MAML sequences include:

(MAML-1; amino acids 19-62):
SEQ ID NO: 2
VMERLRRRIELCRRHHSTCEARYEAVSPERLELERQHTFALHQR
(MAML-2):
SEQ ID NO: 3
IVERLRARIAVCRQHHLSCEGRYERGRAESSDRERESTLQLLSL
(MAML-3):
SEQ ID NO: 4
VVERLRQRIEGCRRHHVNCENRYQQAQVEQLELERRDTVSLYQR
(MAML-1; includes predicted domain for binding
the transcription complex):
SEQ ID NO: 5
HSAVMERLRRRIELCRRHHSTCEARYEAVSPERLELERQHTFALHQRCI
QAKAKRAGKH
(MAML-2; includes predicted domain for binding
the transcription complex):
SEQ ID NO: 6
HSAIVERLRARIAVCRQHHLSCEGRYERGRAESSDRERESTLQLLSLVQ
HGQGARKAGKH
(MAML-3; includes predicted domain for binding
the transcription complex):
SEQ ID NO: 7
AVPKHSTVVERLRQRIEGCRRHHVNCENRYQQAQVEQLELERRDTVSLY
QRTLEQRAKKS
(MAML-1 core)
SEQ ID NO: 8
ERLRRRIELCRRHHST
(MAML-2 core)
SEQ ID NO: 9
ERLRARIAVCRQHHLSC
(MAML-3 core)
SEQ ID NO: 10
ERLRQRIEGCRRHHVN
(MAML-2 fragment):
SEQ ID NO: 11
ERLRARIAVCRQHHLSCEGRYERGRAESS
(MAML-3 fragment):
SEQ ID NO: 21
ERLRQRIEGCRRHHVNCENRYQQAQVEQL

The cross-linked peptides of the present disclosure include at least 10 contiguous amino acids of SEQ ID NOs: 12-20 wherein the side chain of two or more amino acids that are separated by three or seven amino acids is replaced by an internal cross-link. In each case, the amino acids indicated below can be replaced by the corresponding alpha-methyl amino acid. Thus, Leu can be alpha-methyl Leu. The cross-linked peptides of the invention do not include cross-liked peptide comprising any of SEQ ID NO:1-10 wherein in two or more amino acids separated by 3 or 6 amino acids are replaced by an internal cross-link.

Glu₁Arg₂Xaa₃Xaa₄Arg₅Arg₆Xaa₇Xaa₈Xaa₉Xaa₁₀Arg₁₁
Xaa₁₂His₁₃His₁₄Ser₁₅Xaa₁₆
(SEQ ID NO: 12; Related to MAML-1)
Glu₁Arg₂Xaa₃Xaa₄Ala₅Arg₆Xaa₇Xaa₈Xaa₉Xaa₁₀Arg₁₁
Xaa₁₂His₁₃His₁₄Leu₁₅Xaa₁₆Xaa₁₇Xaa₁₈Gly₁₉Arg₂₀
Xaa₂₁Glu₂₂Arg₂₃Gly₂₄Arg₂₅Ala₂₆Glu₂₇Ser₂₈Ser₂₉
(SEQ ID NO: 15; Related to MAML-2)
Glu₁Arg₂Xaa₃Xaa₄Gln₅Arg₆Xaa₇Xaa₈Xaa₉Xaa₁₀Arg₁₁
Xaa₁₂His₁₃His₁₄Val₁₅Xaa₁₆Xaa₁₇Xaa₁₈Asn₁₉Arg₂₀
Xaa₂₁Gln₂₂Gln₂₃Ala₂₄Gln₂₅Val₂₆Glu₂₇Gln₂₈Leu₂₉
(SEQ ID NO: 18; Related to MAML-3)

wherein:

### Xaa₃ is Leu, Trp or Phe;

Xaa₄ is Arg, Lys, Ala, Aib (aminoisobutyric acid);

### Xaa₇ is Ile, Leu, or NorL;

### Xaa₈ is Glu Ala or Aib;

### Xaa₉ is Leu, Trp, Phe, or Tyr;

### Xaa₁₀ is Cys, Phe or Val;

### Xaa₁₂ is Arg, Ala or Aib Xaa₁₆ is Thr or Ala or Aib;

provided that when Xaa₃ is Leu, Xaa₇ is Ile, and Xaa₉ is Leu, Xaa₁₀ is not Cys; and provided that when Xaa₇ is Ile, and Xaa₉ is Leu, and Xaa₁₀ is Cys, Xaa₃ is not Leu; and provided that when Xaa₃ is Leu, and Xaa₉ is Leu, and Xaa₁₀ is Cys, Xaa₇ is not Ile; and provided that when Xaa₃ is Leu, Xaa₇ is Ile, and Xaa₁₀ is Cys, Xaa₉ is not Leu.

Glu₁Arg₂Xaa₃Xaa₄Arg₅Arg₆Xaa₇Xaa₈Xaa₉Xaa₁₀Arg₁₁Xaa₁₂
His₁₃His₁₄Ser₁₅Xaa₁₆Xaa₁₇Xaa₁₈Ala₁₉Arg₂₀Xaa₂₁
(SEQ ID NO: 13; Related to MAML-l)
Glu₁Arg₂Xaa₃Xaa₄A1a₅Arg₆Xaa₇Xaa₈Xaa₉Xaa₁₀Arg₁₁Xaa₁₂
His₁₃His₁₄Leu₁₅Xaa₁₆Xaa₁₇Xaa₁₈Gly₁₉Arg₂₀Xaa₂₁
(SEQ ID NO: 16; Related to MAML-2)
Glu₁Arg₂Xaa₃Xaa₄Gln₅Arg₆Xaa₇Xaa₈Xaa₉Xaa₁₀Arg₁₁Xaa₁₂
His₁₃His₁₄Val₁₅Xaa₁₆Xaa₁₇Xaa₁₈Asn₁₉Arg₂₀Xaa₂₁
(SEQ ID NO: 19; Related to MAML-3)

### Wherein:

### Xaa₃ is Leu, Trp or Phe;

Xaa₄ is Arg, Lys, Ala, Aib (aminoisobutyric);

### Xaa₇ is Ile, Leu, or NorL;

### Xaa₈ is Glu Ala or Aib;

### Xaa₉ is Leu, Trp, Phe, or Tyr;

### Xaa₁₀ is Cys, Phe or Val;

### Xaa₁₂ is Arg, Ala or Aib;

### Xaa₁₆ is Thr, Ala or Aib;

### Xaa₁₇ is Cys, Aib, Ala, or D-pentafluorophenylalanine.;

### Xaa₁₈ is Glu, Ala or Aib.

Glu₁Arg₂Xaa₃Xaa₄Arg₅Arg₆Xaa₇Xaa₈Xaa₉Xaa₁₀Arg₁₁Xaa₁₂
His₁₃His₁₄Ser₁₅Xaa₁₆Xaa₁₇Xaa₁₈Ala₁₉Arg₂₀Xaa₂₁Glu₂₂
Ala₂₃Val₂₄Ser₂₅Pro₂₆Glu₂₇Arg₂₈Leu₂₉
(SEQ ID NO: 14)
Glu₁Arg₂Xaa₃Xaa₄Ala₅Arg₆Xaa₇Xaa₈Xaa₉Xaa₁₀Arg₁₁Xaa₁₂
His₁₃His₁₄Leu₁₅Xaa₁₆Xaa₁₇Xaa₁₈Gly₁₉Arg₂₀Xaa₂₁Glu₂₂
Arg₂₃Gly₂₄Arg₂₅Ala₂₆Glu₂₇Ser₂₈Ser₂₉
(SEQ ID NO: 17; Related to MAML-2)
Glu₁Arg₂Xaa₃Xaa₄Gln₅Arg₆Xaa₇Xaa₈Xaa₉Xaa₁₀Arg₁₁Xaa₁₂
His₁₃His₁₄Val₁₅Xaa₁₆Xaa₁₇Xaa₁₈Asn₁₉Arg₂₀Xaa₂₁Gln₂₂
Gln₂₃Ala₂₄Gln₂₅Val₂₆Glu₂₇Gln₂₈Leu₂₉
(SEQ ID NO: 20; Related to MAML-3)

wherein:

Xaa₃ is Leu, Trp or Phe;

### Xaa₄ is Arg, Lys, Ala or Aib Xaa₇ is Ile, Leu, or NorL;

### Xaa₈ is Glu or Ala or Aib

### Xaa₉ is Leu, Trp, Phe, or Tyr;

### Xaa₁₀ is Cys, Phe or Val;

### Xaa₁₂ is Arg, Ala or Aib

### Xaa₁₆ is Thr or Ala or Aib

### Xaa₁₇ is Cys, Aib, Ala or D-pentafluorophenylalanine.;

### Xaa₁₈ is Glu, Ala or Aib

Xaa₂₁ is Tyr, 1-naphthylalanine, Trp, or 2-naphthylalanine.

In some embodiments the cross-linked peptide is a described above provided that: when Xaa₃ is Leu, Xaa₇ is Ile, and Xaa₉ is Leu, Xaa₁₀ is not Cys; and/or provided that when Xaa₇ is Ile, and Xaa₉ is Leu, and Xaa₁₀ is Cys, Xaa₃ is not Leu; and/or provided that when Xaa₃ is Leu, and Xaa₉ is Leu, and Xaa₁₀ is Cys, Xaa₇ is not Ile; and/or provided that when Xaa₃ is Leu, Xaa₇ is Ile, and Xaa₁₀ is Cys, Xaa₉ is not Leu.

In the cross-linked peptides described herein the alpha carbon of an amino acid at position N can be cross-linked to the alpha carbon of an amino acid at position N+4 by replacing the side chains of both amino acids with an internal cross-link. In the case of peptides have two internal cross links, the alpha carbon of the amino acid at position N can be cross-linked to the alpha carbon of an amino acid at position N+4 by replacing the side chains of both amino acids with an internal cross-link and the alpha carbon of the amino acid at position N+8 can be cross-linked to the alpha carbon of an amino acid at position N+12 by replacing the side chains of both amino acids with an internal cross-link. In the case of so-called stitched peptides having two cross-links in which one amino acid participates in two cross-links, i.e., the alpha carbon of one amino acid is cross-linked to two different amino acids, the alpha carbon of the amino acid at position N can be cross-linked to the alpha carbon of the amino acid at position N+4 and the alpha carbon of the amino acid at position N+4 can also be cross-linked to the alpha carbon of the amino acid at position N+8. This is usually accomplished by replacing the side chain of the amino acid at position N with a cross-link to the alpha carbon to the amino acid at position N+4, replacing each of the side chain and the H of the amino acid a position N+4 with cross links (one to the amino acid at position N and one to the amino acid at position N+8), and replacing the side chain of the amino acid at position N+8 with a cross-link to the alpha carbon of the amino acid at position N+4.

In SEQ ID NO:12 (and 13-20) preferred cross-links are: between Xaa₄ and Xaa₈: between Xaa₈ and Xaa₁₂; between Xaa₁₂ and Xaa₁₆; between Xaa₄ and Xaa₈ and simultaneously between Xaa₈ and Xaa₁₂ (stitched peptide); and between Xaa₈ and Xaa₁₂ and simultaneously between Xaa₁₂ and Xaa₁₆ (stitched peptide).

In one aspect, the present disclosure features a modified polypeptide of Formula (I),

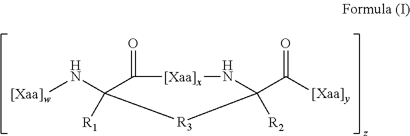

or a pharmaceutically acceptable salt thereof,

wherein;

each R₁ and R₂ are independently H or a C₁ to C₁₀ alkyl (preferably methyl), C₂ to C₁₀ alkenyl, C₂ to C₁₀ alkynyl, arylalkyl, cycloalkylalkyl, heteroarylalkyl, or heterocyclylalkyl;

R₃ is alkylene, alkenylene or alkynylene, or [R₄′-K-R₄]ₙ; each of which is substituted with 0-6 R₅;

R₄ and R₄′ are independently alkylene, alkenylene or alkynylene (e.g., each are independently a C1, C2, C3, C4, C5, C6, C7, C8, C9 or C10 alkylene, alkenylene or alkynylene);

R₅ is halo, alkyl, OR₆, N(R₆)₂, SR₆, SOR₆, SO₂R₆, CO₂R₆, R₆, a fluorescent moiety, or a radioisotope;

K is O, S, SO, SO₂, CO, CO₂, CONR₆,

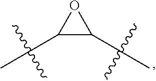 [SMILES: *C1OC1*]

aziridine, episulfide, diol, amino alcohol or

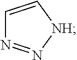

R₆ is H, alkyl, or a therapeutic agent;

n is 2, 3, 4 or 6;

x is an integer from 2-10 (preferably 3 or 6);

w and y are independently an integer from 0-100;

z is an integer from 1-10 (e.g., 1, 2, 3, 4, 5, 6, 7, 8, 9, 10); and

each Xaa is independently an amino acid (e.g., one of the 20 naturally occurring amino acids or any naturally occurring non-naturally occurring amino acid, e.g., a D-amino acid or an alpha-alkyl amino acid (e.g., an alpha-methyl-amino acid));

wherein the polypeptide comprises at least 8 contiguous amino acids of any of SEQ ID NOs 12-20 or a variant thereof, or another polypeptide sequence described herein except that: (a) within the 8 contiguous amino acids of SEQ ID NO:12-20 the side chains of at least one pair of amino acids separated by 3, 4 or 6 amino acids is replaced by the linking group, R₃, which connects the alpha carbons of the pair of amino acids as depicted in Formula I; and (b) the alpha carbon of the first of the pair of amino acids is substituted with R₁ as depicted in formula I and the alpha carbon of the second of the pair of amino acids is substituted with R₂ as depicted in Formula I. Thus, the sequence [Xaa]wL′[Xaa]yL″[Xaa]z, wherein L′ and L″ are amino acids in which the side chains have been replaced by the linking group R₃, comprises at least contiguous amino acids of SEQ ID NO:12-20.

In another aspect, the invention features a modified polypeptide of Formula (II),

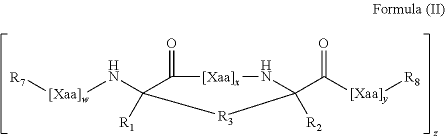

or a pharmaceutically acceptable salt thereof,

wherein;

each R₁ and R₂ are independently H or a C1-C10 alkyl, C2-C10 alkenyl, C2-C10 alkynyl, arylalkyl, cycloalkylalkyl, heteroarylalkyl, or heterocyclylalkyl;

R₃ is C8-C16 alkylene, C8-C16 alkenylene (preferably a C8 alkenylene with a double bond between the 4^{th} and 5^{th} carbons) or C8-C16 alkynylene, or [R₄′-K-R₄]ₙ; each of which is substituted with 0-6 R₅;

R₄ and R₄′ are independently C1-C10 alkylene, C2-C10 alkenylene or C2-C10 alkynylene (e.g., each are independently a C1, C2, C3, C4, C5, C6, C7, C8, C9 or C10 alkylene, alkenylene or alkynylene);

R₅ is halo, alkyl, OR₆, N(R₆)₂, SR₆, SOR₆, SO₂R₆, CO₂R₆, R₆, a fluorescent moiety, or a radioisotope;

K is O, S, SO, SO₂, CO, CO₂, CONR₆,

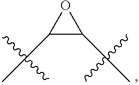 [SMILES: *C1OC1*]

aziridine, episulfide, diol, amino alcohol, or

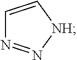

R₆ is H, C1-C10 alkyl, or a therapeutic agent;

x is an integer from 2-10 (preferably 3 or 6);

w and y are independently an integer from 0-100;

z is an integer from 1-10 (e.g., 1, 2, 3, 4, 5, 6, 7, 8, 9, 10); and

each Xaa is independently an amino acid (e.g., one of the 20 naturally occurring amino acids or any naturally occurring non-naturally occurring amino acid);

R₇ is PEG, a tat protein, an affinity label, a targeting moiety, a fatty acid-derived acyl group, a biotin moiety, a fluorescent probe (e.g. fluorescein or rhodamine) linked via, e.g., a thiocarbamate, carbamate, amide, amine, ether or triazole linkage;

R₈ is H, OH, NH₂, NHR₈ₐ, NR₈ₐR_{8b};

wherein the polypeptide comprises at least 14 contiguous amino acids of SEQ ID NOs 12-20 or a variant thereof, or another polypeptide sequence described herein except that: (a) within any of SEQ ID NOs:12-20 the side chains of at least one pair of amino acids separated by 3, 4 or 6 amino acids is replaced by the linking group, R₃ᵢ which connects the alpha carbons of the pair of amino acids as depicted in formula I; and (b) the alpha carbon of the first of the pair of amino acids is substituted with R₁ as depicted in Formula II and the alpha carbon of the second of the pair of amino acids is substituted with R₂ as depicted in Formula II. Thus, the peptide [Xaa]wX[Xaa]yX′[Xaa]x, where [Xaa]w, [Xaa]y, and [Xaa]x are as defined above in Formulas I and II and X and X′ represent amino acids whose side chain has been replaced by a cross-link, can have a sequence corresponding to at least 20 contiguous amino acids of any of SEQ ID NOs: 12-20. Thus, the sequence [Xaa]wL′[Xaa]yL″[Xaa]z, wherein L′ and L″ are amino acids in which the side chains have been replaced by the linking group R₃, comprises at least contiguous amino acids of SEQ ID NO:12-20.

In some embodiments [R₄′-K-R₄]ₙ is

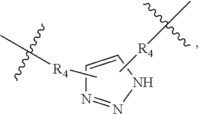

wherein each R₄ is independently a C2-C6 alkyl. In some embodiments R₇ is spermine (—(CH₂)₃NH(CH₂)₃NH(CH₂)₃NH₂)

Also included are peptides having formula IV:

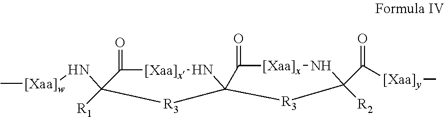

or a pharmaceutically acceptable salt thereof,

wherein;

each R₁ and R₂ are independently H or a C₁ to C₁₀ alkyl (preferably methyl), C₂ to C₁₀ alkenyl, C₂ to C₁₀ alkynyl, arylalkyl, cycloalkylalkyl, heteroarylalkyl, or heterocyclylalkyl;

R₃ is alkylene, alkenylene or alkynylene, or [R₄′-K-R₄]ₙ; each of which is substituted with 0-6 R₅;

R₄ and R₄′ are independently alkylene, alkenylene or alkynylene (e.g., each are independently a C1, C2, C3, C4, C5, C6, C7, C8, C9 or C10 alkylene, alkenylene or alkynylene);

R₅ is halo, alkyl, OR₆, N(R₆)₂, SR₆, SOR₆, SO₂R₆, CO₂R₆, R₆, a fluorescent moiety, or a radioisotope;

K is O, S, SO, SO₂, CO, CO₂, CONR₆,

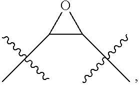 [SMILES: *C1OC1*]

aziridine, episulfide, diol, amino alcohol or

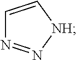

R₆ is H, alkyl, or a therapeutic agent;

x and x′ are independently an integer from 2-10 (preferably 3 or 6; preferably both are 3 or one is 3 and the other is 6 or one is 3 and the other is 6);

w and y are independently an integer from 0-100; and

each Xaa is independently an amino acid (e.g., one of the 20 naturally occurring amino acids or any naturally occurring non-naturally occurring amino acid, e.g., a D-amino acid or an alpha-alkyl amino acid (e.g., an alpha-methyl-amino acid));

wherein the polypeptide comprises at least 8 contiguous amino acids of any of SEQ ID NOs 12-20 or a variant thereof, or another polypeptide sequence described herein except that: (a) within the 8 contiguous amino acids of SEQ ID NO:12-20 the side chains of at least one pair of amino acids separated by 3, 4 or 6 amino acids is replaced by the linking group, R₃, which connects the alpha carbons of the pair of amino acids as depicted in Formula I; and (b) the alpha carbon of the first of the pair of amino acids is substituted with R₁ as depicted in formula I and the alpha carbon of the second of the pair of amino acids is substituted with R₂ as depicted in Formula I.

As noted above, the cross-links can have a variety of positions. Certain examples are depicted below. In these depictions “AA” represents an amino acid side chain and “L” represents the intramolecular cross-link (R₃ in Formulas I-IV)

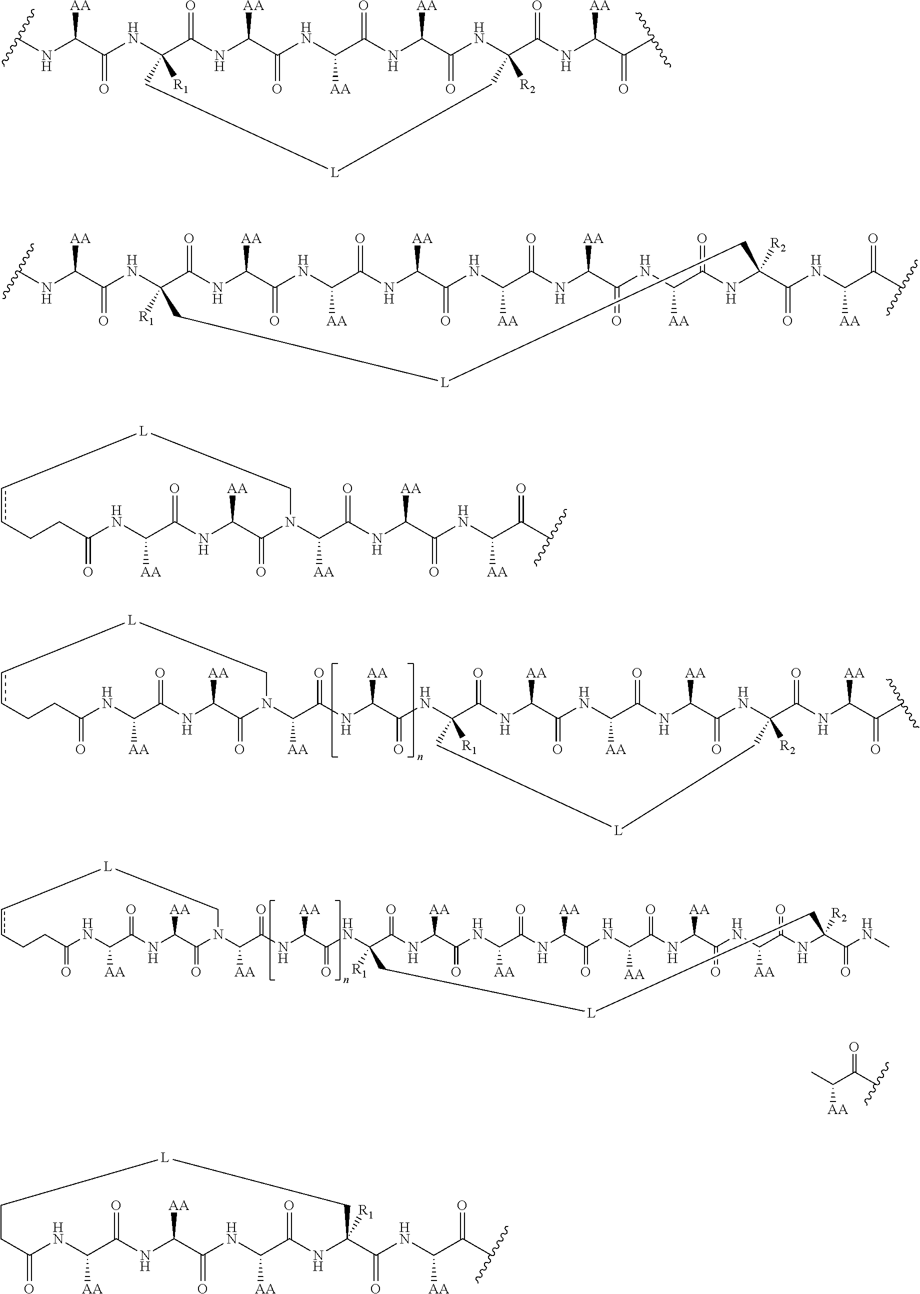

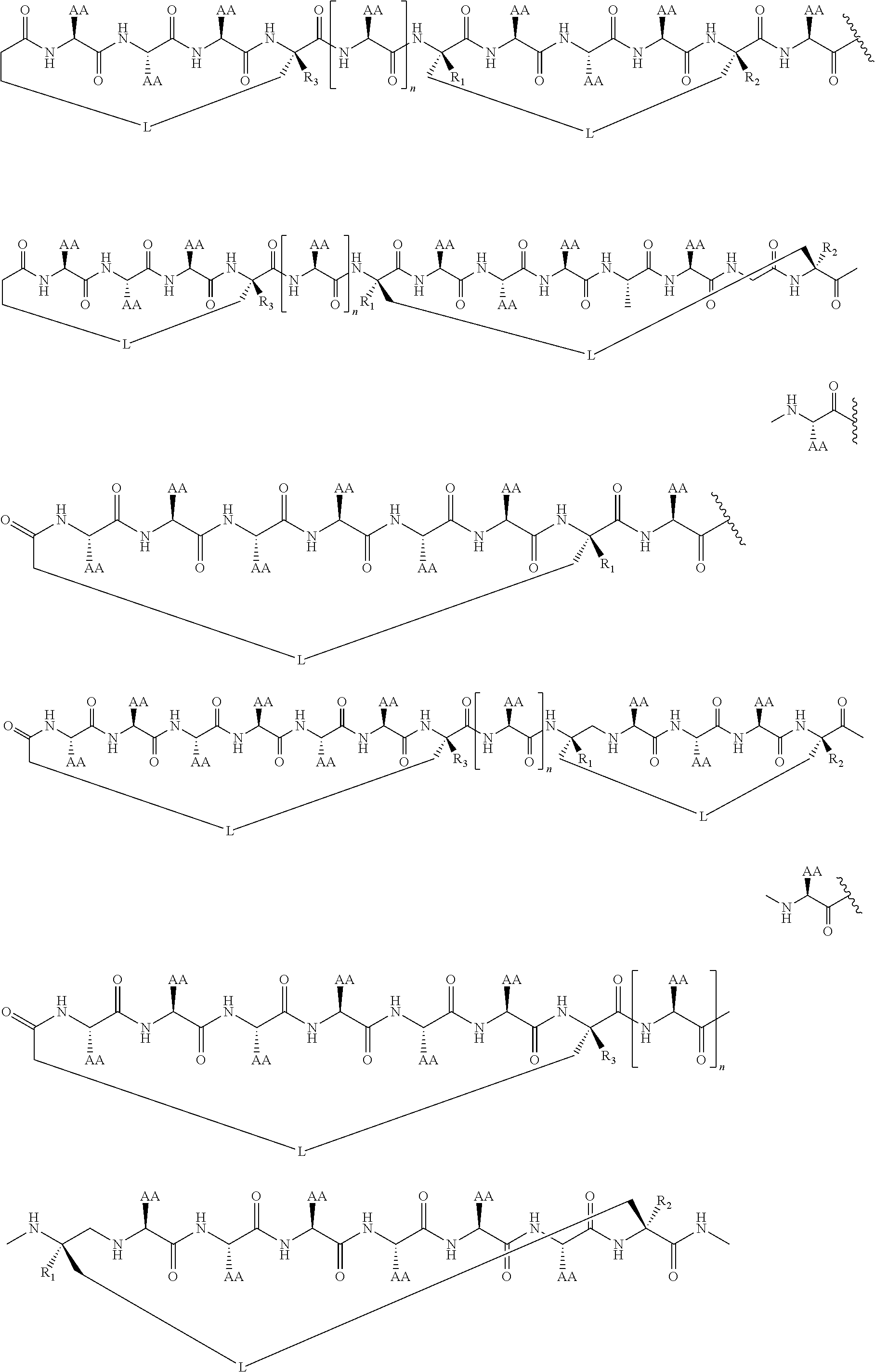

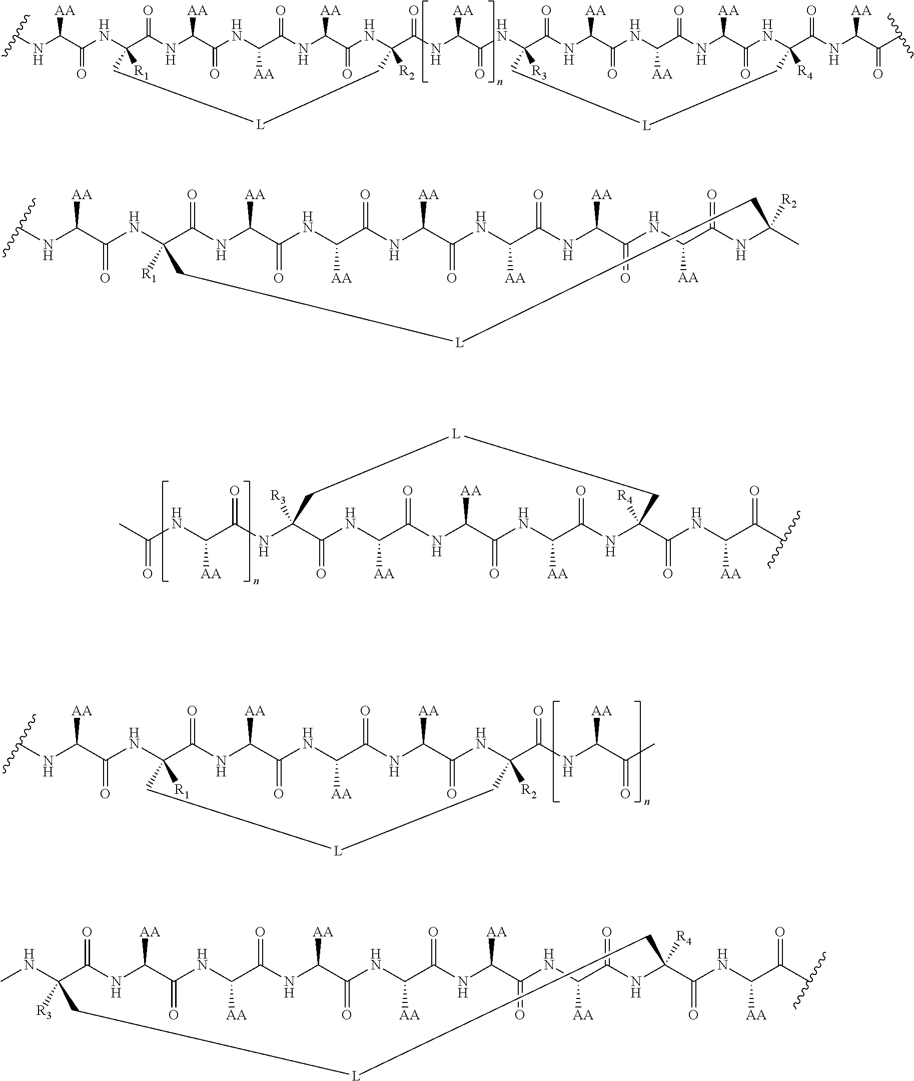

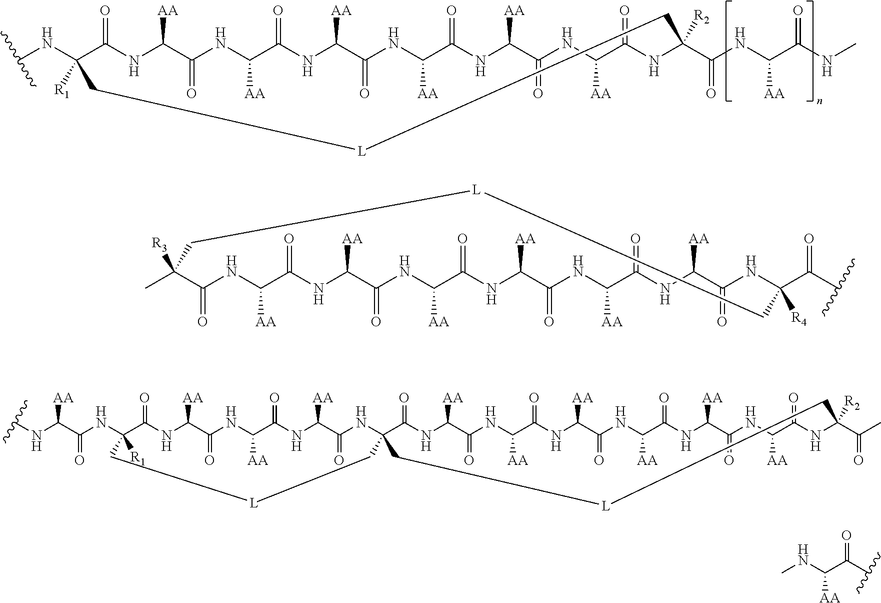

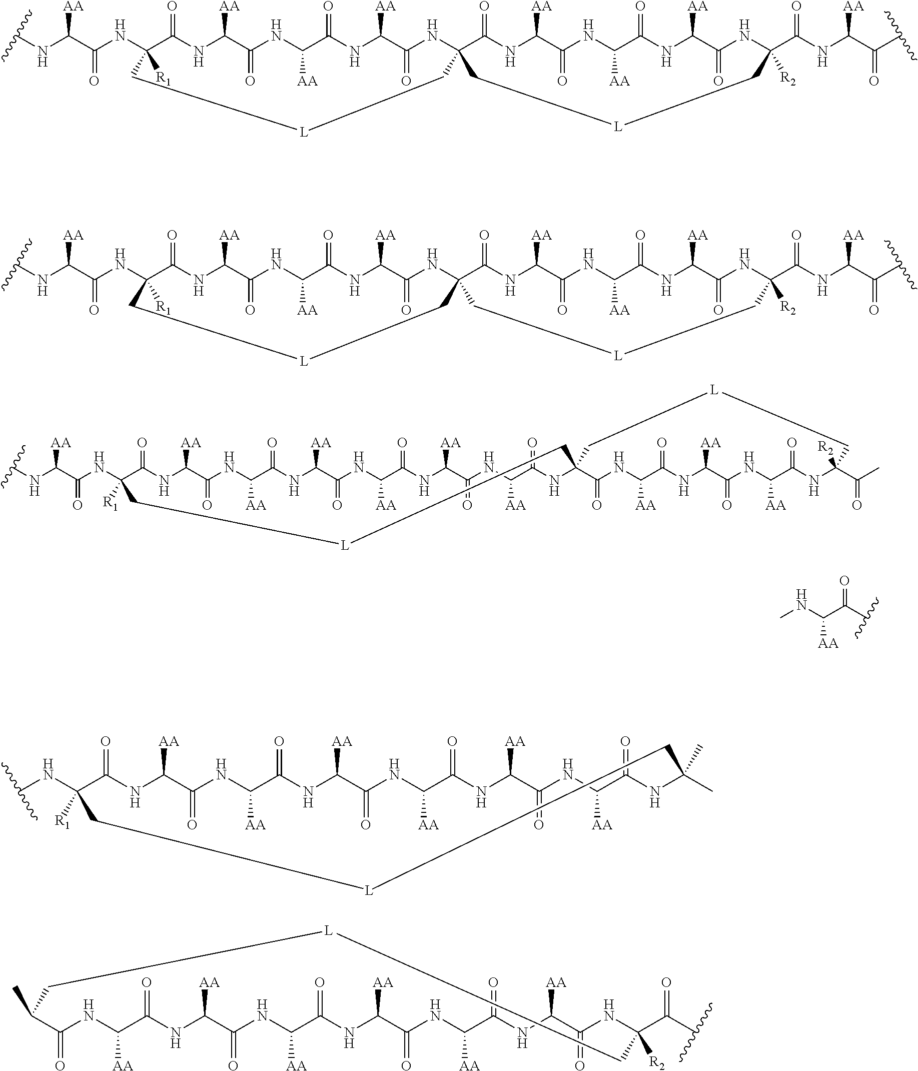

In the case of Formula I or Formula II, the following embodiments are among those disclosed.

In cases where x=2 (i.e., N+3 linkage), R₃ can be a C7 alkylene or alkenylene. Where it is an alkenylene there can one or more double bonds. In cases where x=6 (i.e., i+7 linkage), R₃ can be a C12 or C13 alkylene or alkenylene. Where it is an alkenylene there can one or more double bonds. In cases where x=3 (i.e., i+4 linkage), R₃ can be a C8 alkylene, alkenylene. Where it is an alkenylene there can one or more double bonds.

In the stapled peptides, any position occupied by Gln can be Glu instead and any position occupied by Glu can be Gln instead. Similarly, any position occupied by Asn can be Asp instead and any position occupied by Asp can be Asn instead. In some cases, choice of Asn or Arg and Gln or Glu will depend on the desired charge of the stapled peptide. In many cases it is desirable for the cross-linked peptide to be neutral or have a net positive charge at physiological pH.

In some instances, each w is independently an integer between 3 and 15. In some instances each y is independently an integer between 1 and 15. In some instances, R₁ and R₂ are each independently H or C₁-C₆ alkyl. In some instances, R₁ and R₂ are each independently C₁-C₃ alkyl. In some instances, at least one of R₁ and R₂ are methyl. For example R₁ and R₂ are both methyl. In some instances R₃ is alkyl (e.g., C₈ alkyl) and x is 3. In some instances, R₃ is C₁₁ alkyl and x is 6. In some instances, R₃ is alkenyl (e.g., C₈ alkenyl) and x is 3. In some instances x is 6 and R₃ is C₁₁ alkenyl. In some instances, R₃ is a straight chain alkyl, alkenyl, or alkynyl. In some instances R₃ is —CH₂—CH₂—CH₂—CH═CH—CH₂—CH₂—CH₂—. In some instances R₃ is —CH₂—CH₂—CH₂—CH₂—CH₂—CH₂—CH═CH—CH₂—CH₂—CH₂—. In some instances R₃ is —CH₂—CH₂—CH₂—CH═CH—CH₂—CH₂—CH₂—CH₂—CH₂—CH₂—.

In certain instances, the two alpha, alpha disubstituted stereocenters (alpha carbons) are both in the R configuration or S configuration (e.g., N, N+4 cross-link), or one stereocenter is R and the other is S (e.g., N, N+7 cross-link). Thus, where Formula I is depicted as

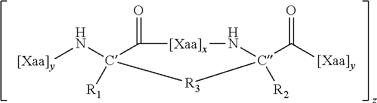

the C′ and C″ disubstituted stereocenters can both be in the R configuration or they can both be in the S configuration, for example when x is 3. When x is 6, the C′ disubstituted stereocenter is in the R configuration and the C″ disubstituted stereocenter is in the S configuration. When x is 2, the C′ disubstituted stereocenter is in the R configuration and the C″ disubstituted stereocenter is in the S configuration. The R₃ double bond may be in the E or Z stereochemical configuration. Similar configurations are possible for the carbons in Formula II corresponding to C′ and C″ in the formula depicted immediately above.

In some instances R₃ is [R₄-K-R₄′]ₙ; and R₄ and R₄′ are independently alkylene, alkenylene or alkynylene (e.g., each are independently a C1, C2, C3, C4, C5, C6, C7, C8, C9 or C10 alkylene, alkenylene or alkynylene

In some instances, the polypeptide includes an amino acid sequence which, in addition to the amino acids side chains that are replaced by an intermolecular cross-link, have 1, 2, 3, 4 or 5 amino acid changes in any of SEQ ID NOs:1-21 (e.g., SEQ ID NOs; 12-20).

The cross-link can include an alkyl, alkenyl, or alkynyl moiety (e.g., C₅, C₈ or C₁₁ alkyl or a C₅, C₈ or C_{1I} alkenyl, or C₅, C₈ or C₁₁ alkynyl). The cross-linked amino acid can be alpha disubstituted (e.g., C₁-C₃ or methyl). [Xaa]_{y} and [Xaa]_{w} are peptides that can independently comprise at least 1, 2, 3, 4, 5, 6, 7, 8, 9, 10, 11, 12, 13, 14, 15, 16, 17, 18, 19, 20, 25 or more contiguous amino acids (preferably 2 or 5 contiguous amino acids) of a variant MAML1, 2 or 3 peptide (e.g., any of SEQ ID NOs:12-20) and [Xaa]ₓ is a peptide that can comprise 3 or 6 contiguous amino acids of acids of a variant MAML1, 2 or 3 peptide.

The peptide can comprise 8, 9, 10, 11, 12, 13, 14, 15, 16, 17, 18, 19, 20, 25, 30, 35, 40, 45, 50 amino acids of a variant MAML1, 2 or 3 peptide. The amino acids are contiguous except that one or more pairs of amino acids separated by 3 or 6 amino acids are replaced by amino acid substitutes that form a cross-link, e.g., via R₃. Thus, at least two amino acids can be replaced by cross-linked amino acids or cross-linked amino acid substitutes. Thus, where formula I is depicted as

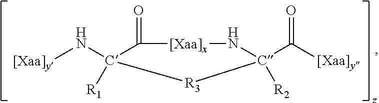

[Xaa]_{y′}, [Xaa]ₓ and [Xaa]_{y″} can each comprise contiguous polypeptide sequences from the same or different variant MAML1, 2 and 3 peptides. The same is true for Formula II.

The peptides can include 10 (11, 12, 13, 14, 15, 16, 17, 18, 19, 20, 21, 22, 23, 24, 25, 30, 35, 40, 45, 50 or more) contiguous amino acids of a variant MAML1, 2 or 3 polypeptide described herein wherein the alpha carbons of two amino acids that are separated by three amino acids (or six amino acids) are linked via R₃, one of the two alpha carbons is substituted by R₁ and the other is substituted by R₂ and each is linked via peptide bonds to additional amino acids.

In some instances the polypeptide acts as an inhibitor of Notch complex formation. In some instances, the polypeptide also includes a fluorescent moiety or radioisotope or a moiety that can chelate a radioisotope (e.g., mercaptoacetyltriglycine or 1,4,7,10-tetraazacyclododecane-N,N′,N″,N′″-tetraacetic acid (DOTA)) chelated to a radioactive isotope of Re, In or Y). In some instances, R₁ and R₂ are methyl; R₃ is C₈ alkyl, C₁₁ alkyl, C₈ alkenyl, C₁₁ alkenyl, C₈ alkynyl, or C₁₁ alkynyl; and x is 2, 3, or 6. In some instances, the polypeptide includes a PEG linker, a tat protein, an affinity label, a targeting moiety, a fatty acid-derived acyl group, a biotin moiety, a fluorescent probe (e.g. fluorescein or rhodamine) or another bio-active molecule to recruit enzymatic machinery, including: small molecules that bind and recruit ubiquitin ligases (nutlin, SAH-p53-8); histone deacetylase proteins and complexes (SIN3 alpha-helix, SAHA) or co-activator proteins (MLL alpha-helix, VP16 alpha-helix) or others.

Also described herein is a method of treating a subject including administering to the subject any of the compounds described herein. In some instances, the method also includes administering an additional therapeutic agent, e.g., a chemotherapeutic agent.

The peptides may contain one or more asymmetric centers and thus occur as racemates and racemic mixtures, single enantiomers, individual diastereomers and diastereomeric mixtures and geometric isomers (e.g. Z or cis and E or trans) of any olefins present. All such isomeric forms of these compounds are expressly included in the present invention. The compounds may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are included as are all crystal forms.

Amino acids containing both an amino group and a carboxyl group bonded to a carbon referred to as the alpha carbon. Also bonded to the alpha carbon is a hydrogen and a side-chain. Suitable amino acids include, without limitation, both the D- and L-isomers of the 20 common naturally occurring amino acids found in peptides (e.g., A, R, N, C, D, Q, E, G, H, I, L, K, M, F, P, S, T, W, Y, V (as known by the one letter abbreviations)) as well as the naturally occurring and unnaturally occurring amino acids prepared by organic synthesis or other metabolic routes. The table below provides the structures of the side chains for each of the 20 common naturally-occurring amino acids. In this table the “—” at right side of each structure is the bond to the alpha carbon.

| | Single | Three | |
|---|---|---|---|
| Amino acid | Letter | Letter | Structure of side chain |
| Alanine | A | Ala | CH₃— |
| Arginine | R | Arg | HN═C(NH₂)—NH—(CH₂)₃— |
| Asparagine | N | Asn | H₂N—C(O)—CH₂— |
| Aspartic acid | D | Asp | HO(O)C—CH₂— |
| Cysteine | C | Cys | HS—CH₂— |
| Glutamine | Q | Gln | H₂N—C(O)—(CH₂)₂— |
| Glutamic acid | E | Glu | HO(O)C—(CH₂)₂— |
| Glycine | G | Gly | H— |
| Histidine | H | His | 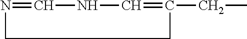 |
| Isoleucine | I | Ile | CH₃—CH₂—CH(CH₃)— |
| Leucine | L | Leu | (CH₃)₂—CH—CH₂— |
| Lysine | K | Lys | H₂N—(CH₂)₄— |
| Methionine | M | Met | CH₃—S—(CH₂)₂— |
| Phenylalanine | F | Phe | Phenyl-CH₂— |
| Proline | P | Pro | 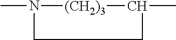 |
| Serine | S | Ser | HO—CH₂— |
| Threonine | T | Thr | CH₃—CH(OH)— |
| Tryptophan | W | Trp | 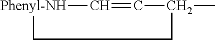 |
| Tyrosine | Y | Tyr | 4-OH-Phenyl-CH₂— |
| Valine | V | Val | CH₃—CH(CH₂)— |

A “non-essential” amino acid residue is a residue that can be altered from the wild-type sequence of a polypeptide (without abolishing or substantially altering its activity. An “essential” amino acid residue is a residue that, when altered from the wild-type sequence of the polypeptide, results in abolishing or substantially abolishing the polypeptide activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The symbol “ [symbol] ” when used as part of a molecular structure refers to a single bond or a trans or cis double bond.

The term “amino acid side chain” refers to a moiety attached to the α-carbon in an amino acids. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an alpha di-substituted amino acid).

The term “polypeptide” encompasses two or more naturally occurring or synthetic amino acids linked by a covalent bond (e.g., a amide bond). Polypeptides as described herein include full length proteins (e.g., fully processed proteins) as well as shorter amino acids sequences (e.g., fragments of naturally occurring proteins or synthetic polypeptide fragments). The term “variant MAML-1 peptide” includes SEQ ID NOs: 12-14. The term “variant MAML-2 peptide” includes SEQ ID NOs: 15-17. The term “variant MAML-3 peptide” includes SEQ ID NOs: 18-20.

The term “halo” refers to any radical of fluorine, chlorine, bromine or iodine. The term “alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 20 (inclusive) carbon atoms in it. The term “alkylene” refers to a divalent alkyl (i.e., —R—).

The term “alkenyl” refers to a hydrocarbon chain that may be a straight chain or branched chain having one or more carbon-carbon double bonds in either Z or E geometric configurations. The alkenyl moiety contains the indicated number of carbon atoms. For example, C₂-C₁₀ indicates that the group may have from 2 to 10 (inclusive) carbon atoms in it. The term “lower alkenyl” refers to a C₂-C₈ alkenyl chain. In the absence of any numerical designation, “alkenyl” is a chain (straight or branched) having 2 to 20 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂-C₁₀ indicates that the group may have from 2 to 10 (inclusive) carbon atoms in it. The term “lower alkynyl” refers to a C₂-C₈ alkynyl chain. In the absence of any numerical designation, “alkynyl” is a chain (straight or branched) having 2 to 20 (inclusive) carbon atoms in it.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, 1,2,3-triazolyl, 1,2,4-triazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, aziridinyl, oxiryl, thiiryl, morpholinyl, tetrahydrofuranyl, and the like.

The term “substituents” refers to a group “substituted” on an alkyl, cycloalkyl, aryl, heterocyclyl, or heteroaryl group at any atom of that group. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, azido, and cyano groups.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 | Modeling the NOTCH1-MAML1-CSL ternary complex (NTC). a) Schematic of NTC assembly and activation of target gene expression. Stabilized alpha-helical peptides derived from MAML1 (SAHMs) mimicking the N-terminal helix of MAML1 target the ANK1-CSL interface and prevent target gene activation. b) Molecular modeling of the NTC. Left—RMSD (Å) of the NTC along the 35 ns MD simulation. Right—Decomposition of individual residue binding energies in the NTC by MMGBSA in Amber10. The dominant negative fragment of MAML1 (dnMAML1, residues 13-74), ANK domain of NOTCH1 (ANK1) and CSL are showing in magenta, red and blue, respectively. Residues identified as the strongest contributors to complex stability are highlighted in yellow (top residues 1-9) and cyan (residues 10-18) and are represented as sticks in dnMAML1 and surfaces for ANK1 and CSL. Bottom—Average (Ave, kcal/mol) binding free energy for residues highlighted in the NTC structure. Residues in red are the highest scoring residues for their respective protein subunit. c) Binding free energy (kcal/mol) for all residues in the contact region of dnMAML1 (residues 16-70) as determined by BFED. d) Mastermind homolog sequence alignment. Residues 20-41 of human MAML1 are aligned with sequences from H. sapiens, M. musculus, D. melanogaster, X. laevis, C. elegans and D. rerio. Orange, residues conserved among all species; Green, conserved substitutions; blue, semi-conserved substitutions. e&f) Left—Backbone RMSD (A) of the unmodified MAML1 (21-36) peptide (e) and SAHM1 (f) along a 20 ns MD simulation. Right—Overlay of MAML1 (21-36) peptide (e) and SAHM1 (f) snapshots extracted every 1 ns from 20 ns MD simulation trajectories. g) Left—Schematic of computational structure-activity relationships workflow for BFED calculations for SAHM point-mutants. Right—Calculated MMGBSA ΔΔG values for SAHM peptides containing the indicated point mutation are shown relative to the unmodified MAML1 (E21-T36) peptide (WT).

FIG. 2 | Analysis of dnMAML1-RAMANK1-CSL complex formation and ALPHAscreen assay development. a) Schematic of the ALPHAscreen proximity assay. Incubation of a synthetic, biotinylated-dnMAML1 peptide with equimolar GST-RAMANK1 and CSL protein leads to the formation of the active NTC in solution and proximal association of streptavidin-coated donor beads with anti-GST-conjugated acceptor beads. Donor bead excitation at 680 nm produces singlet oxygen, which selectively initiates a luminescent cascade in bound acceptor beads. b) Synthetic, biotinylated dnMAML1 (Bio-sdnMAML1, residues 16-70) was synthesized with an N-terminal diethylene glycol linker and biotin tag. The chromatogram and mass spectrum of the HPLC-purified peptide is shown. c-e) SPR binding of immobilized Bio-sdnMAML1 (c), Bio-nts-dnMAML1 (d) and Bio-SAHM1 (e) to dilutions of soluble, equimolar RAMANK1 and CSL. Black curves represent reference-cell normalized sensogram data and red curves denote a kinetic fit to a two-step kinetic model. Binding constants derived from this fit are shown. kₒₙ, association rate; k_{off}, dissociation rate; K_{d}, dissociation constant; RU, response units. f) Titration matrix of Bio-sdnMAML1 and GST-RAMANK1-CSL binding partners. g) ALPHAscreen signals under optimal conditions (40 nM of Bio-sdnMAML1, GST-RAMANK1 and CSL) yielded robust binding only in the presence of all NTC partners. h) Unlabeled dnMAML1 and SAHM1 peptides competed with Bio-sdnMAML1 for GST-RAMANK1-CSL binding relative to DMSO control. 1) Competitive ALPHAscreen assays for previously reported unmodified and stapled SAHM peptides. Data are shown as mean±s.e.m. of duplicate or triplicate measurements for matrix titrations (f) and peptide competition assays (h), respectively.

FIG. 3 | Design and biochemical characterization of SAHM analog peptides. a) Panel of SAHM analogs used in MD simulations containing point mutations at positions 23, 27, 29 and 30. b) ALPHAscreen competition assay screen of point-mutant analogs as well as previously characterized peptides (1 μM) to compete with Bio-sdnMAML1 for GST-RAMANK1-CSL binding relative to DMSO. c-g) MD snapshots of high-scoring SAHM point mutants containing the C30F (C), L29W (D), L29Y (E), I27N_{L} (F) and L23F (G) point mutations. Relevant contacts are highlighted and discussed in the text. h & i) Views of additional contacts to the ANK1 domain (red) and CSL (blue) mediated by C37-Y41 (green) and E42-L49 (orange) residues of dnMAML1 in the human NTC X-ray structure (PDB Accession: 2F8X). ALPHAscreen competition values shown represent the mean±s.e.m.

FIG. 4 | Biochemical characterization and SAR of SAHM analog peptides. a) Structures and ALPHAscreen NTC competition assay IC₅₀ values of analog peptides containing natural mutation combinations in the E21-T36 scaffold. b) Structures and ALPHAscreen NTC competition assay IC₅₀ values of analog peptides containing natural mutation combinations in the E21-Y41 and E21-L49 extended stapled peptide scaffolds. c) Structures and ALPHAscreen NTC competition assay IC₅₀ values of analog stapled peptides containing non-natural amino acids. The blue “B₅” residues in the stitched peptides SAHM1-29 and SAHM1-30 correspond to a bis-pentenyl glycine derivative (see Suppl. FIG. 3). Competition curves (Right) represent the mean±s.e.m. of duplicate experiments fitted to a three-parameter sigmoidal dose-response curve in Prizm 5. ALPHAscreen IC₅₀ values shown represent the 95% confidence interval (C.I.) of the mean. d) MD snapshot of SAHM1-56 (magenta ribbon view) bound to the ANK1-CSL complex (white surface view) with the L23W, L29Y and C30F mutant side chains highlighted and shown as sticks. e) Overlaid MD snapshots of the E21-L49 scaffold analog SAHM1-80 bound to the ANK1-CSL complex before (green) and after (blue and magenta ribbon view) MD energy minimization. d) MD snapshot of SAHM1-62 (green ribbon view) bound to the ANK1-CSL complex (white surface view) with the L23W, L29Y, C30F, C37F_{f} and Y41Nₚ₁ mutant side chains and hydrocarbon staple highlighted and shown as sticks.

FIG. 5 | SAHM analogs inhibit NOTCH 1-dependent transcription and T-ALL cell proliferation. a) Correlation plot of ALPHAscreen IC₅₀ values and normalized inhibition of a NOTCH 1-driven CSL-luciferase reporter (15 μM, 18 h, relative to DMSO control) for all SAHM analog peptides. b) Dose-dependent inhibition of the NOTCH1-driven dual-luciferase reporter assay by SAHM1 and optimized peptides from the E21-T36 and E21-Y41 SAHM scaffolds. c) Effect of SAHM analog peptides on the proliferation of human T-ALL cell lines previously shown to be sensitive (HPB-ALL and SUPT-1) or predominantly resistant (Jurkat) to NOTCH1 inhibition. Cells were treated with SAHM analogs (20 μM) or DMSO vehicle for 72 h and normalized viability was determined by measuring cellular ATP content with the Cell Titer Glo assay. d) Effect of SAHM1 analogs as in (c) at 10 and 20 μM for 72 h. Data represent mean±s.e.m. from triplicate experiments.

FIG. 6 | Olefin-containing “S₅” and “B₅” amino acids used for synthesis of single turn i, i+4 stapled peptides and two-turn stitched i, i+4+4 stabilized peptides. Residues were incorporated into stapled peptides by conventional SPPS, followed by ring-closing olefin metathesis with Grubbs I catalyst.

FIG. 7 | Structures of bio-sdnMAML1 (a), Ac-sdnMAML1 (b) and bio-nt-sdnMAML1 (c).

FIG. 8 | Graphical representation of reporter gene assay correlation data presented in FIG. 5a. U2OS cells co-transfected with ΔEGFΔLNR-NOTCH1 construct, CSL-Firefly luciferase reporter and Renilla-luciferase reporter were treated with analog stapled peptides (15 μM, 18-24 h) or DMSO vehicle alone. Shown is the normalized mean reporter signal relative to DMSO alone for each analog peptide.

### DETAILED DESCRIPTION

Described below is a molecular dynamics (MD) computational model of the Notch transcriptional complex (NTC). This model was used to explore the global stability of the NTC and the contributions of all residues involved in the protein-protein interfaces of dnMAML1, ANK1 and CSL. Also described below is the use of these models in combination with biochemical assays measuring NTC complex formation. iterative medicinal chemistry approaches and cell-based assays to design cross-linked MAML1 peptides, including one that are more potent than SAHM.

Described below are various internally cross-linked alpha helical domain polypeptides related to human MAML1 (and MAML-2 and MAML-3). The polypeptides include an internal cross-link between two non-natural amino acids (i.e., two amino acids whose side chains have been replaced by the cross-link) that significantly enhances the alpha helical secondary structure of the polypeptide. Generally, the cross-link (sometimes referred to as staple) extends across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . Xaa₁, Xaa₂, Xaa₃, Xaa₄, Xaa₅, Xaa₆, Xaa₇, Xaa₃, Xaa₉ . . . (wherein “ . . . ” indicates the optional presence of additional amino acids), cross-links between Xaa and Xaa₄, or between Xaa₁ and Xaa₅, or between Xaa₁ and Xaa₃ are useful as are cross-links between Xaa₂ and Xaa₅, or between Xaa₂ and Xaa₆, or between Xaa₂ and Xaa₉, etc. The polypeptides can include more than one crosslink within the polypeptide sequence to either further stabilize the sequence or facilitate the stabilization of longer polypeptide stretches. If the polypeptides are too long to be readily synthesized in one part, independently synthesized, cross-linked peptides can be conjoined by a technique called native chemical ligation (Bang, et al., J. Am. Chem Soc. 126:1377).

Described herein are stabilized alpha-helix of MAML1 (SAH-MAML1) peptides that exhibit affinity for the ICN1-CSL complex, and, in contrast to a corresponding unmodified (non-cross-linked) MAML1 peptide, more readily enter cells mechanism.

α,α-Disubstituted non-natural amino acids containing olefinic side chains of varying length can synthesized by known methods (Williams et al. 1991 J. Am. Chem. Soc. 113:9276; Schafmeister et al. 2000 J. Am. Chem Soc. 122:5891). For peptides where an i linked to i+7 staple is used (two turns of the helix stabilized) either one S5 amino acid and one R8 is used or one S8 amino acid and one R5 amino acid is used. R8 is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin.

Methods for preparing cross-linked peptides in which a single amino acid participates in two cross-links are described in US 2010/184645, hereby incorporated by reference.

### Molecular Dynamics Simulation of the NTC

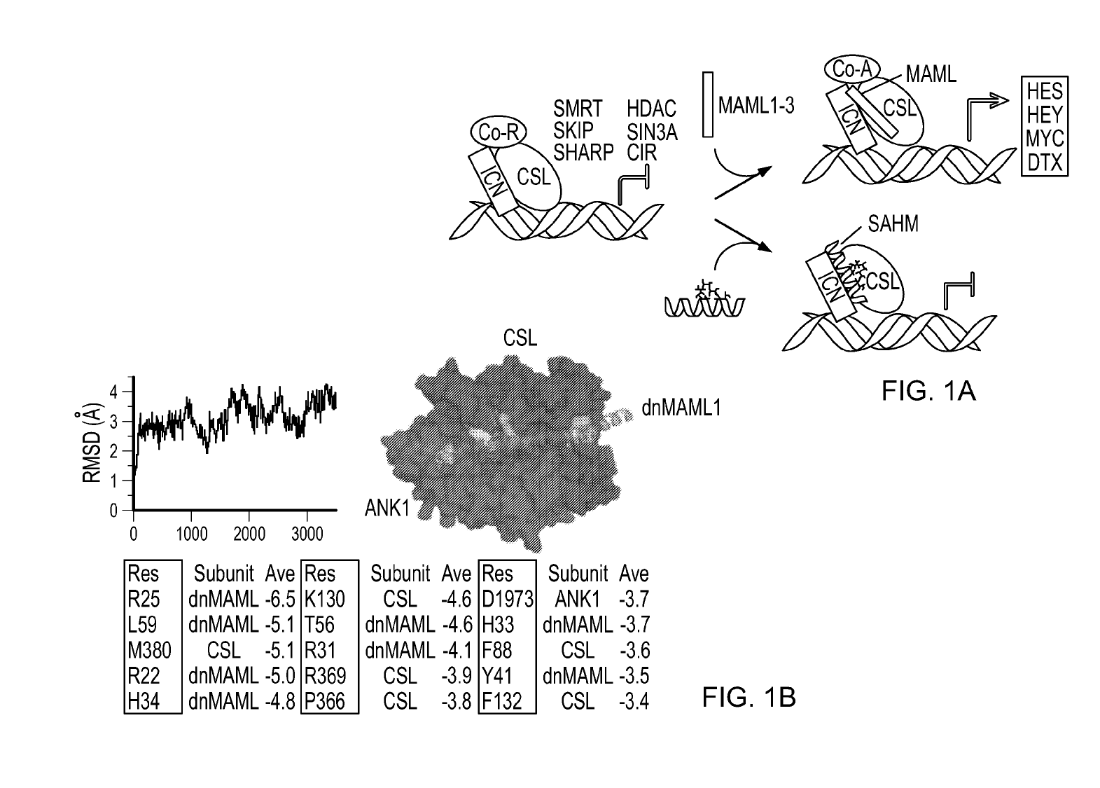

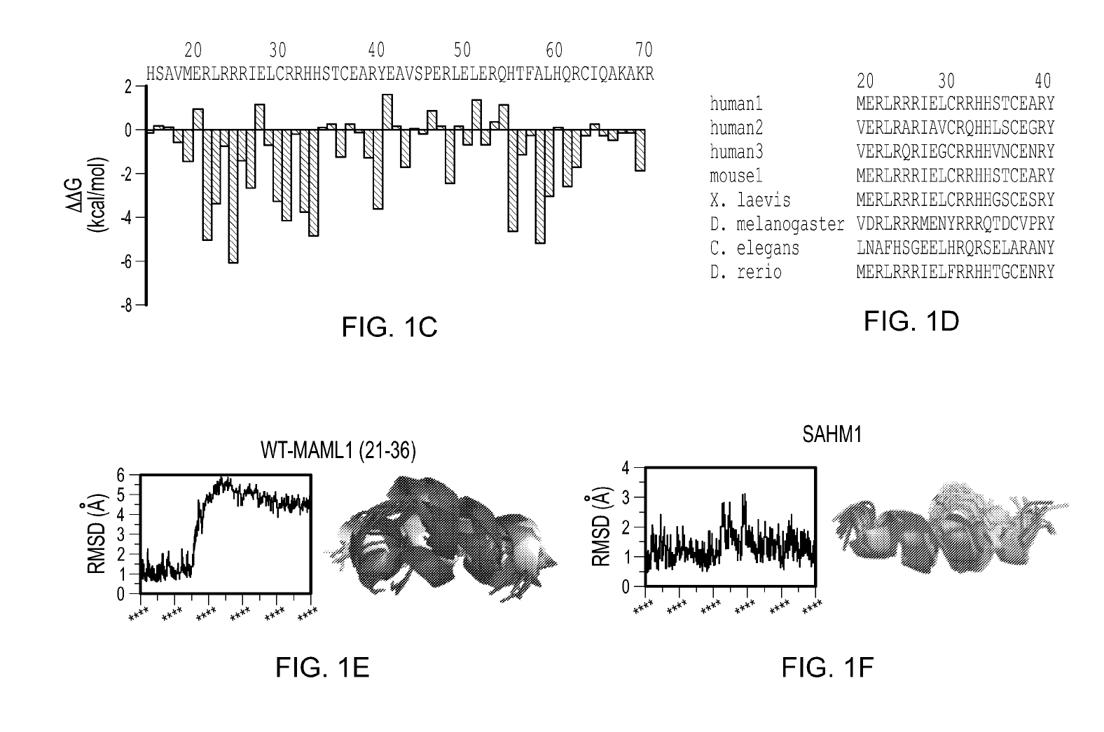

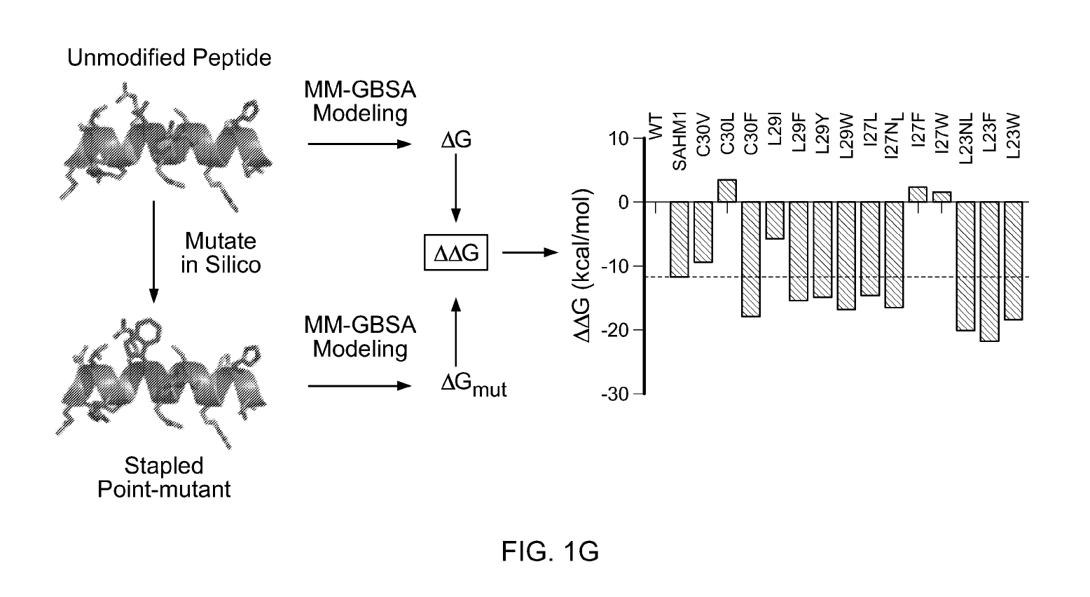

MD simulation of the NTC (dnMAML1-ANK1-CSL) converged after around 5 ns, as evidenced by the relative stabilization of the complex RMSD after this time point (FIG. 1b, left). The protein-peptide binding interaction is composed of a number of weak interactions between pairs of residues in dnMAML1 and the shallow groove at the interface of ANK1-CSL (FIG. 1 b, right). The residues that contribute the most to the binding free energy of an interaction, so called “hot-spots” in the protein-peptide interface, are spatially clustered in some protein-protein interactions (e.g. the p53-MDM2 interface) and are more diffuse along a larger binding interface in others. Our screen of dnMAML1 stapled peptide fragments and published mutagenesis experiments indicate that a majority of the critical contacts are contained in the N-terminal helix, however the extent to which the binding energy is distributed through the interface is largely unknown. In an effort to identify the energy contribution of each residue in dnMAML1 to NTC complex stability, we employed a computational binding free-energy decomposition (BFED) analysis. A similar method is computational alanine scanning (CAS), which computes the change in free energy upon mutating a given residue to alanine. Mutation to alanine can induce significant conformational changes and thus perturb the binding system, which cannot be accounted for by CAS. On the other hand, the BFED method calculates both backbone and side chain energy contributions and does not introduce the perturbation of alanine mutation. We thus applied BFED method to our system based on the snapshots extracted from the converged MD trajectory (5-35 ns).

From the N-terminus to P46, which introduces a kink to an otherwise continuous helix, dnMAML1 binds a surface made up of both ANK1 and CSL. While from P46 to the C-terminus, dnMAML1 only interacts with CSL (FIG. 1b, right). Table 1 lists the top 15 residues that contribute the most to the dnMAML1-ANK1-CSL binding free energies (FIG. 1b, right). We find that 10 (in bold) out of the top 15 hot-spot residues are located between dnMAML1 (N-terminal to P46) and the ANK1-CSL interface, which indicates that this region is more important for binding. Top ranking residues outside of the N-terminal helix cluster around an interaction between a hydrophobic cleft on CSL with L59 and T56 in dnMAML1. Top residues in the N-terminal helix include a cluster of arginines (R22, R25 & R31) in dnMAML1 that form stable salt bridges with D1973 and E2009 in ANK1 and E378 in CSL. Other important residues in this stretch include two histidines (H34 & 1-133) and one tyrosine (Y41), whose van der Waals energy term dominates the free energy of binding. Previous experiments reported that the dual R25E/R22E dnMAML1 mutant or D1973R ANK1 mutant prevented the formation of the NTC complex by gel shift assays⁴⁵. Furthermore, we have previously reported that a stapled peptide, SAHM1-D1, containing R22E/R26E mutations showed diminished activity in numerous assays³⁷. In agreement with these findings, our calculations revealed R25 and D1973 (colored in red in FIG. 1b) as the most important residues in dnMAML1 and ANK1, respectively. The model also predicted M380 (colored in red in FIG. 1b) as the key residue in CSL, which interacts with I27, C30, R31 and H34 in dnMAML1.

FIG. 1c shows the BFED contribution of each residue in dnMAML1, where negative values indicate critical interactions and small or positive values represent unimportant or deleterious interactions, respectively. These calculations recapitulate the results of our reported stapled peptide screen, with the majority of critical contacts contained in the stretch from E21 to T36 used to generate SAHM1. The high concentration of binding energy contribution in this region provides an explanation for the high degree of conservation in this peptide stretch of Mastermind orthologues from numerous species (FIG. 1d). Importantly, these calculations also highlight numerous residues that are involved in binding but may be underutilized, which in this stretch of dnMAML1 include L23, I27, L29 and C30. Mutation of these residues to natural or non-natural amino acids has the potential to generate more potent and specific stapled peptide inhibitors of the NOTCH complex.

### Molecular Dynamics Simulation of Unmodified and Stapled MAML Peptides

We next sought to develop a molecular dynamics model that accurately depicted stapled peptides and could be used to inform the design of SAHM analogues targeting the NTC. Multiple published reports have employed MD simulations to study stapled peptides, however these have primarily been concerned with the effect of the hydrocarbon staple on peptide stability and helicity^{47,48.} We are unaware of any reports that have successfully employed MD simulation to quantitatively inform stapled peptide binding and develop SAR parameters for analogs. To first evaluate the degree to which the E21-T36 dnMAML1 peptide [MAML1 (21-36)] remains helical in our calculations, we performed MD simulations on the corresponding helical structure extracted from the NOTCH complex. We also made the corresponding stapled peptide analogue (SAHM1) by mutating E28 and R32 to the i→i+4 ligated α,α-disubstituted “S₅” amino acids (FIG. 6). 20 ns MD simulations were carried out for MAML1 (21-36) and SAHM1 in explicit solvent using similar parameters as the NTC simulations. The MD trajectories revealed that MAML1 (21-36) loses its α-helical structure after approximately 6-9 ns (FIG. 1e) while SAHM1 retains most of its helical content along the entire 20 ns MD simulation with a little chaos in the C-terminal serine-threonine stretch (FIG. 1f). During the simulation of MAML1 (21-36), the salt bridge between E28 & R32 is disrupted and the backbone hydrogen bonds were lost, which caused the peptide to unfold. Conversely, the central hydrocarbon staple in SAHM1 conserved the helical turn in the middle of the peptide while E21 made transient salt bridges with either R24 or R25. These qualitative results are in agreement with previous circular dichroism measurements comparing the helicity of MAML1 (21-36) and SAHM1³⁷.

Simulations and BFED Calculations of SAHM1 Analogues with ANK1-CSL

Guided by these MD models, we next aimed to determine whether improved SAHM1 analogues could be designed. Analogue peptides containing the non-natural amino acid linker were built based on the initial X-ray structure (PDBid: 2f8x) and MD simulations were run by replacing dnMAML1 with SAHM1 analogues. The mutated natural/non-natural residues (including the staples) were built in Maestro 8.5. Parameters for the creation of non-natural residues are detailed in Experimental Methods. Conformational searches of each new NOTCH complex with the mutated amino acids were performed using Macromodel followed by energy minimization. Mixed torsional/low mode Monte Carlo in Macromodel was applied by allowing the mutated residues to move freely and restraining the surrounding residues within 4 Å by a constant of 200 ( ) and keeping other residues fixed. The conformation with the lowest energy was used as the starting structure for MD simulation with the same parameter settings used for the NTC simulations. 16 ns MD simulations were carried out for each new complex with different SAHM1 analogues. Snapshots were extracted along the converged MD trajectories and MMGBSA scores were calculated to compare the relative binding affinities of SAHM1 analogues to the ANK1-CSL complex.

The results of the aforementioned NTC MD simulations indicated that residues L23, I27, L29 & C30 do not contribute as strongly to dnMAML1 binding free energy. which suggested that these residues might be mutated to make stronger interactions with ANK1-CSL. To test this premise, we designed a focused library of analog peptides containing hydrophobic point mutations at these positions. Analysis or MD trajectories and MMGBSA scores for each peptide was used to determine whether each mutation was favorable or not and which were the best mutation(s) for each position. MMGBSA scores were calculated relative to the unmodified MAML1 (21-36) peptide and are shown in FIG. 1g. Notably, mutations of C30 and L23 to larger aromatic side chains (phenylalanine and tryptophan, respectively) appeared to have the greatest effect on the MMGBSA score. Mutations to L29 and I27 had positive effects in some cases (L29F/Y/W, 127L) and were deleterious in others (L291, I27F/W). Overall, these calculations supported the notion that optimized interactions could be imparted through these mutations. Thus we endeavored to develop robust biochemical assays measuring NTC assembly and to then test this series of analogs. The structural rationale for the effects of these mutations will be discussed below and compared alongside the results of biochemical studies.

### ALPHAscreen Assay Development and NTC Biochemistry

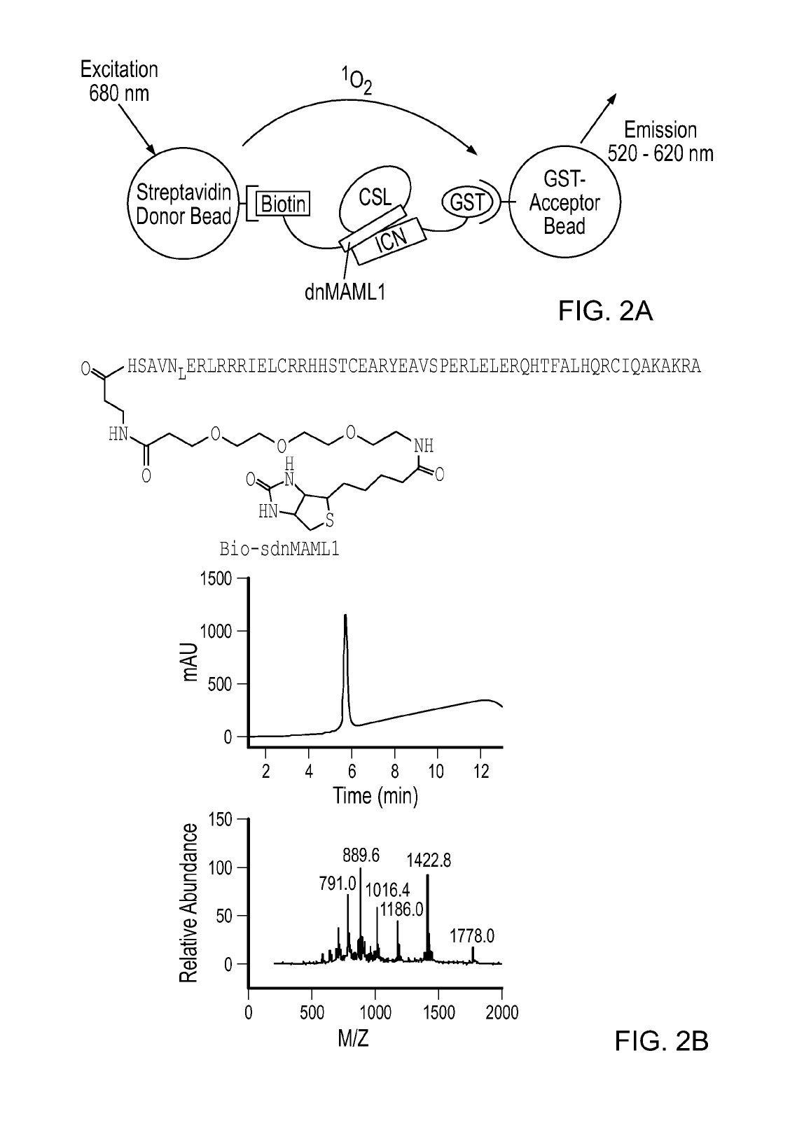

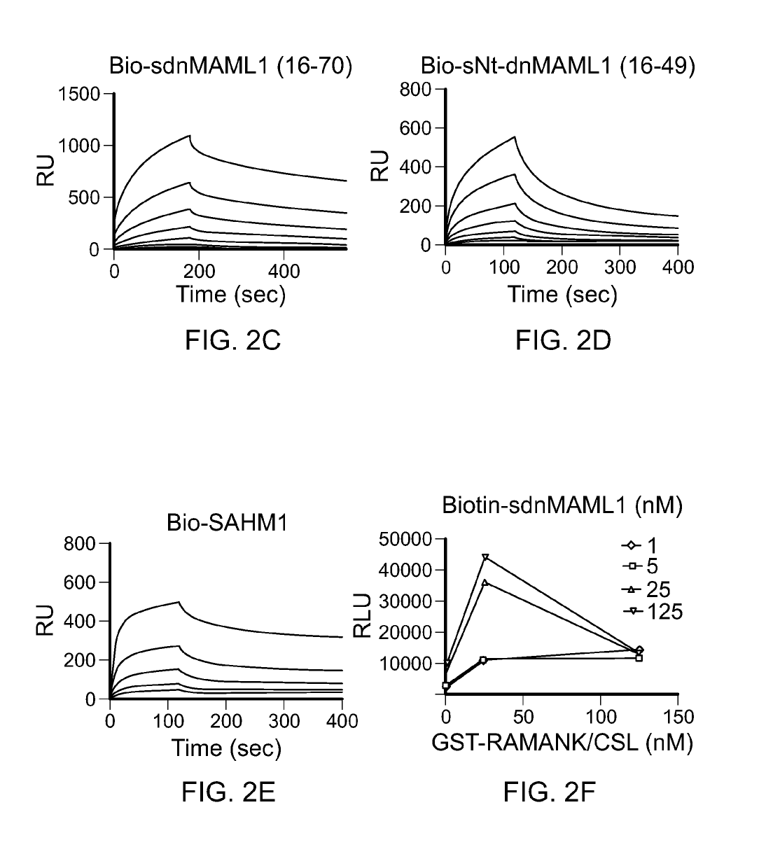

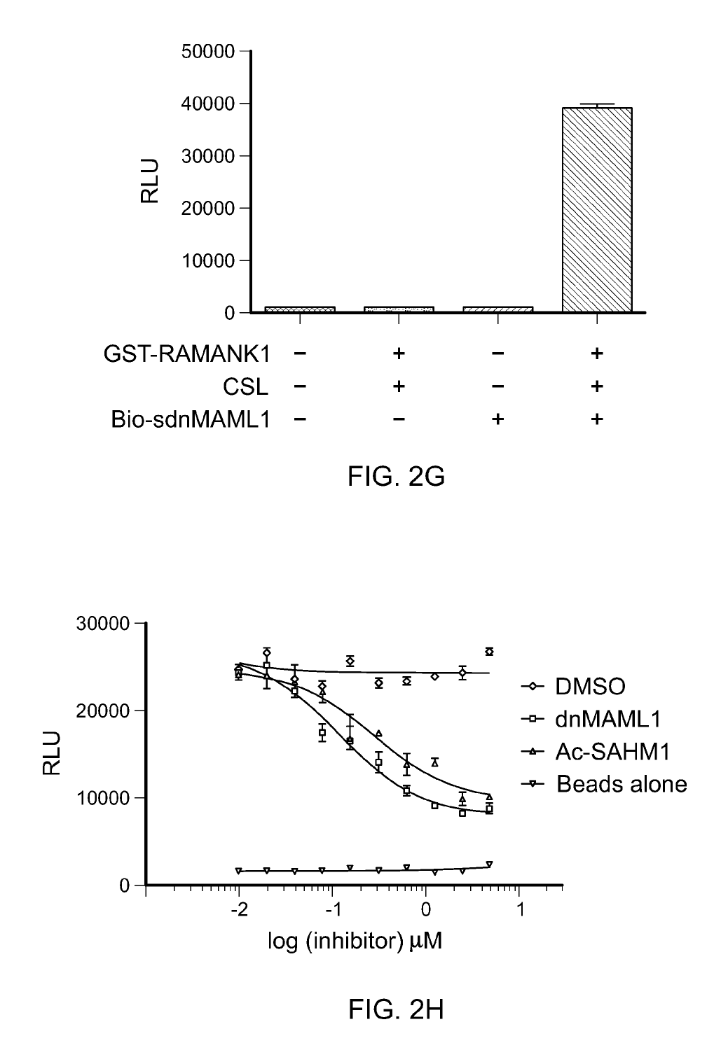

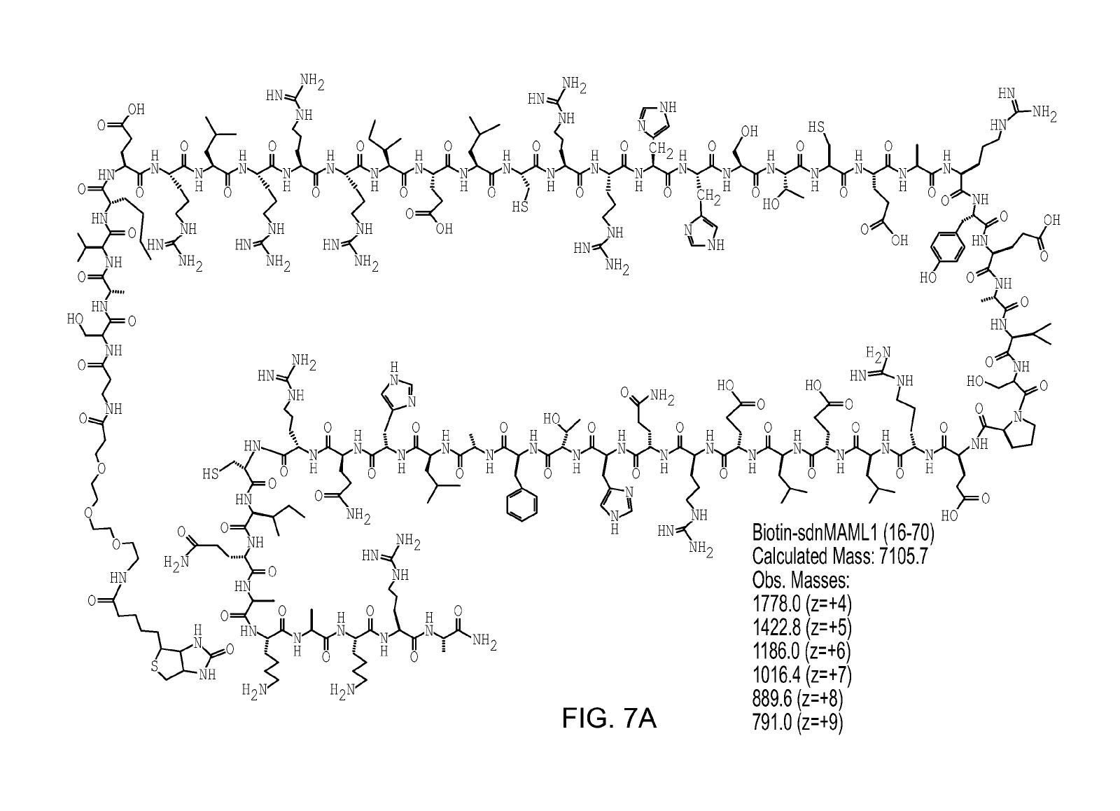

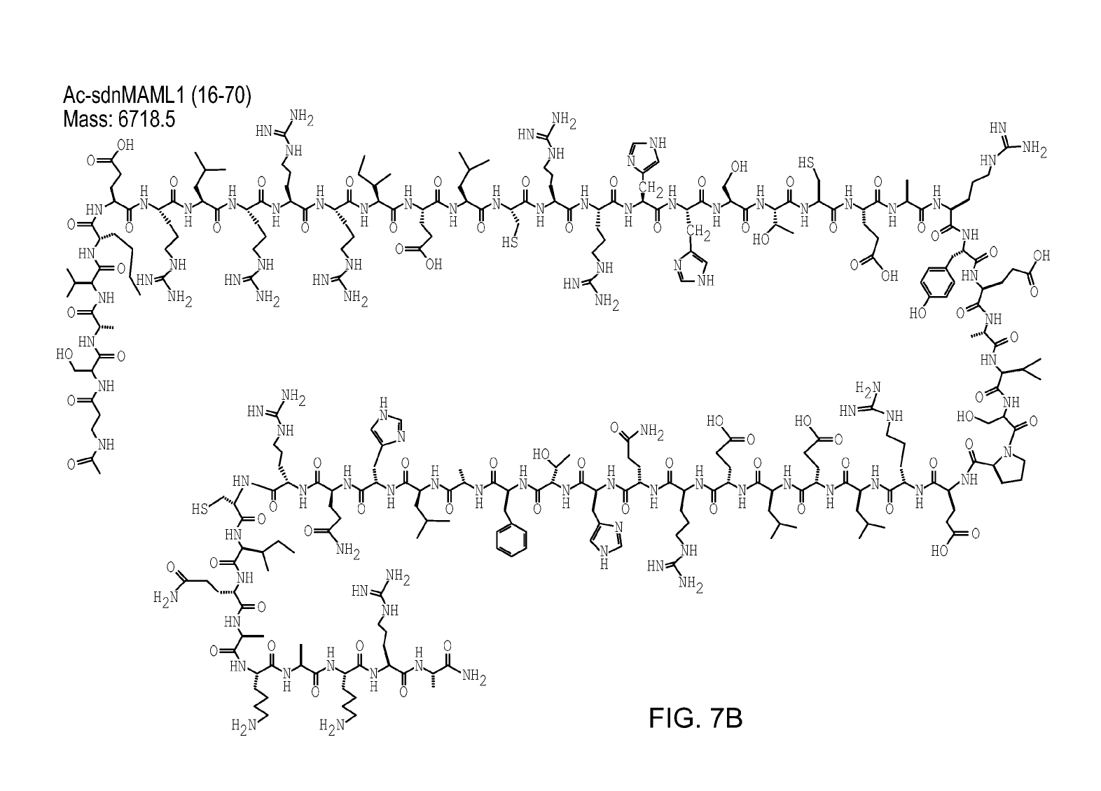

The biophysics of NTC assembly has been studied using relatively low-throughput assays including electrophoretic-mobility shift assays²⁴, isothermal titration calorimetry⁴⁹, and various immunoprecipitation strategies. More recently, Del Bianco et al. reported the use of a FRET-based system measuring the proximity of a donor fluorophore-labeled ANK protein to an acceptor-labeled oligonucleotide upon NTC assembly, which allowed determination of relative equilibrium constants for the entire complex⁴⁵. We also reported the use of surface plasmon resonance (SPR) and fluorescence polarization assays measuring the association of NTC components with each other and with stapled peptides³⁷. To date however, there are no high-throughput assays reported that quantitatively measure dnMAML1-NOTCH-CSL complex formation. Here we introduce a robust, homogenous assay for measuring the binding of dnMAML1 to NOTCH-CSL heterodimers using ALPHAscreen technology. The ALPHAscreen technology (ALPHA meaning amplified luminescence proximity homogenous assay) employs functionalized beads approximately 200 nm in diameter to detect the association of cognate binding partners in solution^{50,51}. Laser excitation (680 nm) of donor beads releases a flow of singlet oxygen, which due to a discrete half-life, will diffuse approximately 200 nm. Acceptor beads that have been proximally localized through a binding interaction will utilize singlet oxygen in a luminescent cascade releasing an emission at lower wavelength (520-620 nm). As shown in FIG. 2a, this assay was configured to detect the association of a synthetic biotinylated dnMAML1 peptide with a complex of CSL and GST-labeled RAMANK1 (the RAM and ANK domains constitute the minimal subunits of ICN for CSL binding). To enable this assay format, we first developed methods to synthesize and purify fully synthetic dnMAML1 polypeptides (sdnMAML1, residues 16-70), which by conventional methods is beyond the size limits of solid-phase peptide synthesis (SPPS, FIG. 2b). The use of microwave-assisted peptide synthesis and a slightly altered SPPS protocol readily afforded the biotinylated sdnMAML1 peptide (bio-sdnMAML1, and others with alternative N-terminal modifications) at greater than 95% purity by LCMS analysis (FIG. 2b, FIG. 7).

SPR was used to measure the binding kinetics between the immobilized bio-sdnMAML1 peptide and equimolar RAMANK1-CSL complexes, which confirmed high-affinity binding (K_{D}=0.04 μM, FIG. 2c). To our knowledge, these experiments represent the first reported affinity for dnMAML1 to any component of the NTC. To determine whether the N-terminal helix alone retains the ability to bind RAMANK1-CSL, as our previous results and MD calculations would suggest, we also measured the affinity of bio-nt-sdnMAML1 (residues 16-45) by SPR (FIG. 2d, FIG. 6). The N-terminal dnMAML1 peptide was found to bind RAMANK1-CSL (K_{D}=0.4 μM), although not as strongly as bio-sdnMAML1 or bio-SAHM1 (K_{D}=0.1 μM, FIG. 2d,e).

To determine the optimal conditions for the NTC ALPHAscreen assay, a titration matrix of various GST-RAMANK1-CSL and bio-sdnMAML1 concentrations was tested. These experiments revealed dose-dependent increases in the luminescent signal up to a maximum of approximately 45,000 c.p.s. with binding components in the range of 10-100 nM (FIG. 20. Importantly, a characteristic “hook effect” was observed at higher concentrations of protein, representing the point where the GST-labeled protein surpasses the binding capacity of the ALPHAscreen beads and becomes inhibitory. Under optimal conditions and binding partner concentrations (40 nM of all partners), this assay was shown to produce excellent signal-to-noise ratios (˜30-fold) and was specific to the presence of all binding partners (FIG. 2g). Titration of unlabeled Ac-sdnMAML1 peptide or AcW-SAHM1 into a pre-incubated complex of bio-sdnMAML1-GSTRAMANK1-CSL resulted in dose-dependent dissociation of the complex and signal decrease (FIG. 2h). Taken together, these results support the generation of a robust, high-throughput biochemical assay for the interrogation of NTC assembly, which is ideally suited for screening NTC inhibitors.

### SAHM Analogue SAR Studies

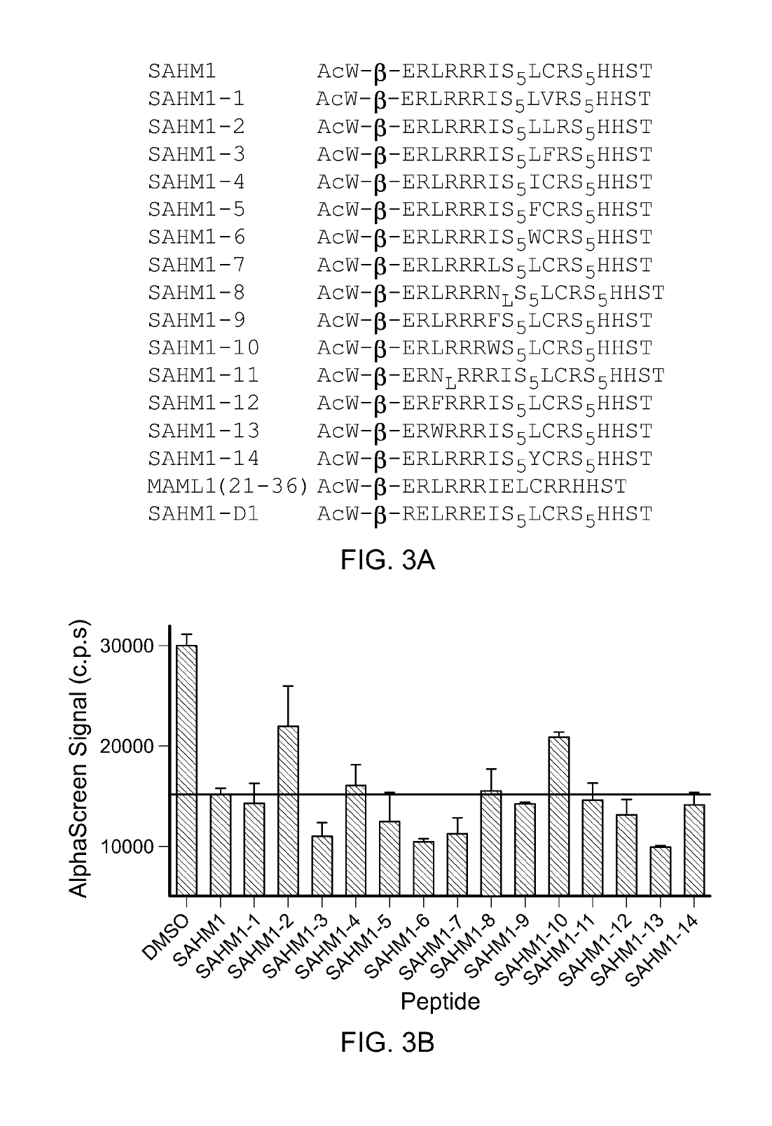

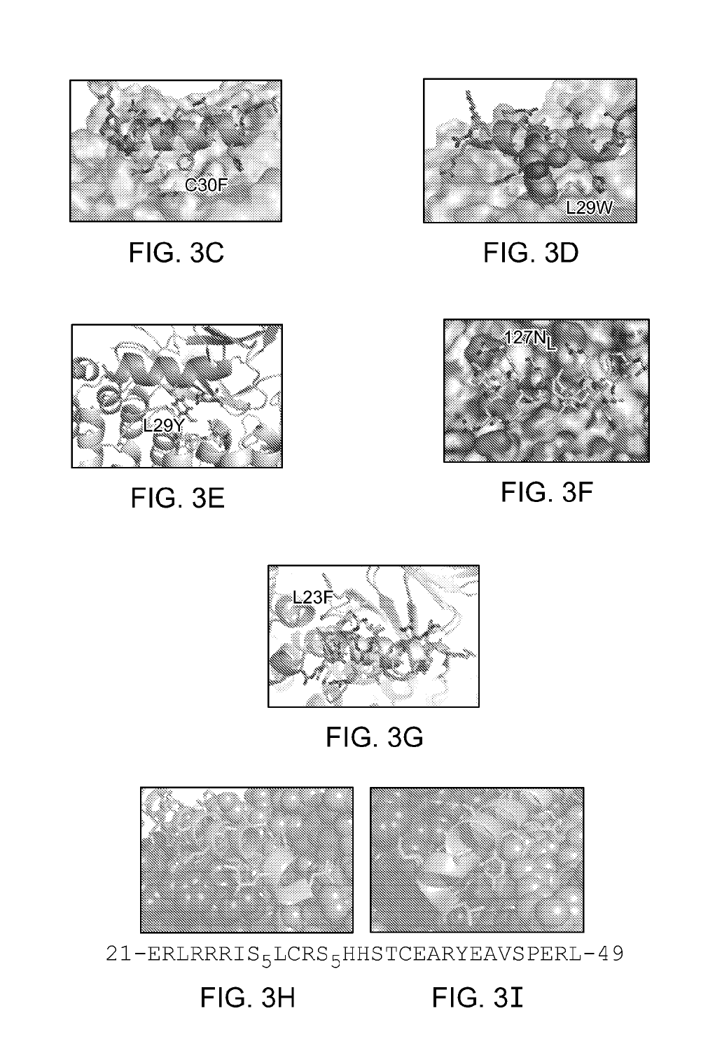

To correlate the relevance of our MD calculations, the series of point-mutant SAHM analogues presented in FIG. 1g was synthesized and profiled by competitive ALPHAscreen (FIG. 3a). In general, the observed inhibitory activities were in good agreement with the MD predictions. Peptides substituted at C30 with either Val or Phe showed greater complex inhibition compared to SAHM1, while the C30L compound was less potent (FIG. 3b). These results mirrored our calculations and visual inspection of MD snapshots with the C30F mutant (SAHM1-3) revealed stable interactions between F30 and M356, L388 and N349 in CSL. Additionally, F30 also induces a loop in ANK1 to move toward CSL, creating an interaction with A2007 (FIG. 3c). The cavity around L29 is quite large and polar and inspection of MD snapshots did not reveal any obvious effects for L291 or L29F mutations, however L29F did improve the MMGBSA score. Mutation of L29 to tryptophan (SAHM1-6), appeared to promote hydrophobic interactions with the side chains of A2007 & L2006 and the backbone of N2041 in ANK1. R382 in CSL also moved closer to W29 to form a potential cation-pi interaction (FIG. 3d). The L29Y (SAHM1-14) mutant also appeared to form this interaction with R382 in CSL as well as hydrogen-bonds with the side chain amide of N2041 and backbone carbonyls of N2040 and V2039 in ANK1 (FIG. 3e). Overall mutations at L29 alone did not have a strong effect on peptide potency, although our modeling results indicated that L29Y and L29W mutations should improve binding, thus these mutations were explored in later combination mutant analogs.

Mutation of 127 to leucine (SAHM1-7) resulted in increased inhibitory activity relative to SAHM1 (FIG. 3b). Conversely, mutation of 127 to the methionine isostere norleucine (NO or phenylalanine was not found to significantly improve potency while mutation to the larger amino acid tryptophan was deleterious for NTC inhibition. These data are consistent with our computational calculations in principle, however MD snapshots predicted that the 127N_{L} mutant would be more potent than its leucine isomer (FIG. 1g). MD snapshots revealed that both the leucine and norleucine side chains more effectively engaged a hydrophobic pocket on CSL surrounded by V354, M356, M380 and V390, leading to improved MMGBSA scores (FIG. 3f). The largest improvements to MMGBSA score and competitive ALPHAscreen potency were generated by mutations of L23. In our MD simulations, introduction of L23F or L23W into SAHM1 was found to induce a conformational change in a flexible loop containing residues N349 to M356 in CSL. Translation of this flexible region improved contacts to the L23F/W as well as I27 and R24 in MD snapshot containing mutant peptides (FIG. 3g). Likewise, increased hydrophobic bulk at this position resulted in iterative decreases in ALPHAscreen competition (SAHM1-11 to SAHM1-13), with SAHM1-13 being the most potent single mutant relative to SAHM1 (FIG. 3b).

Overall these results represented a general agreement between the mutant-NTC MD simulation and biochemical potency against NTC formation, thus validating the use of our stapled peptide-NTC MD model as a strategy for the design of analogue inhibitors.

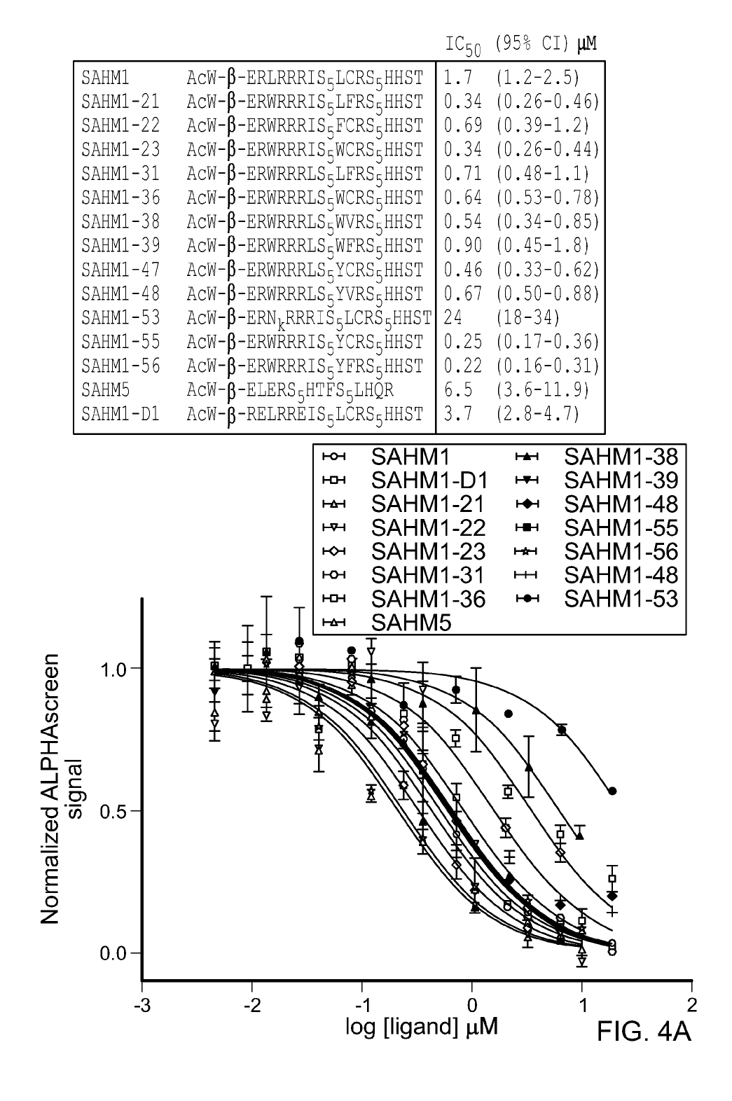

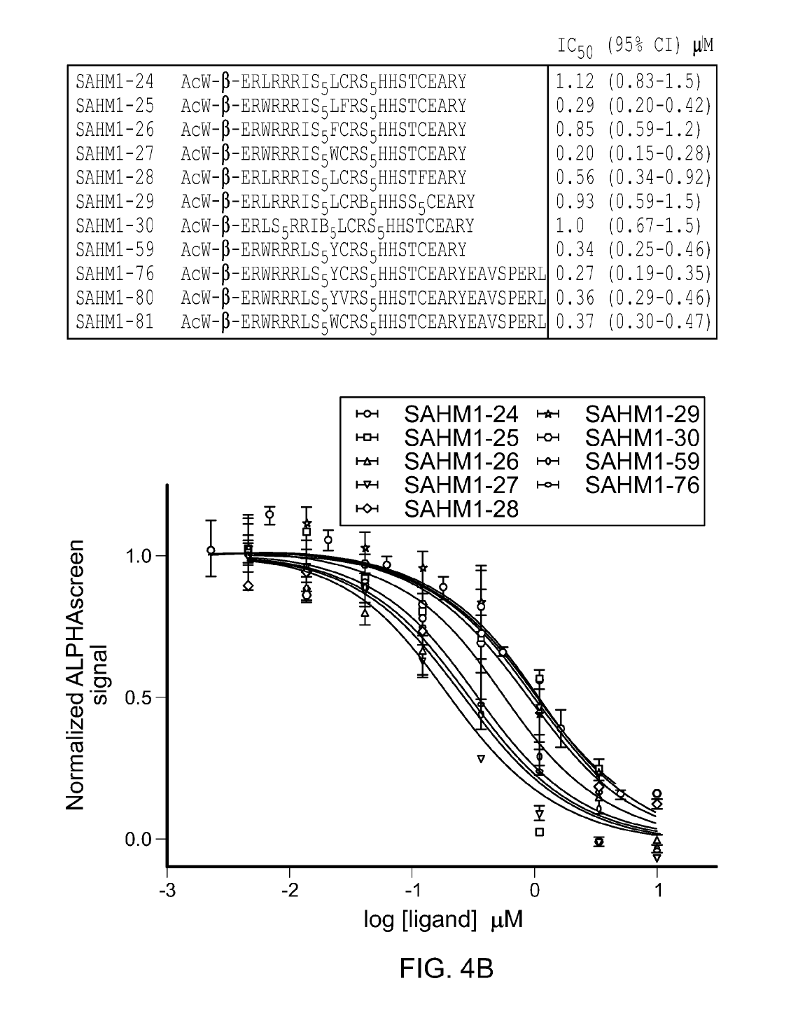

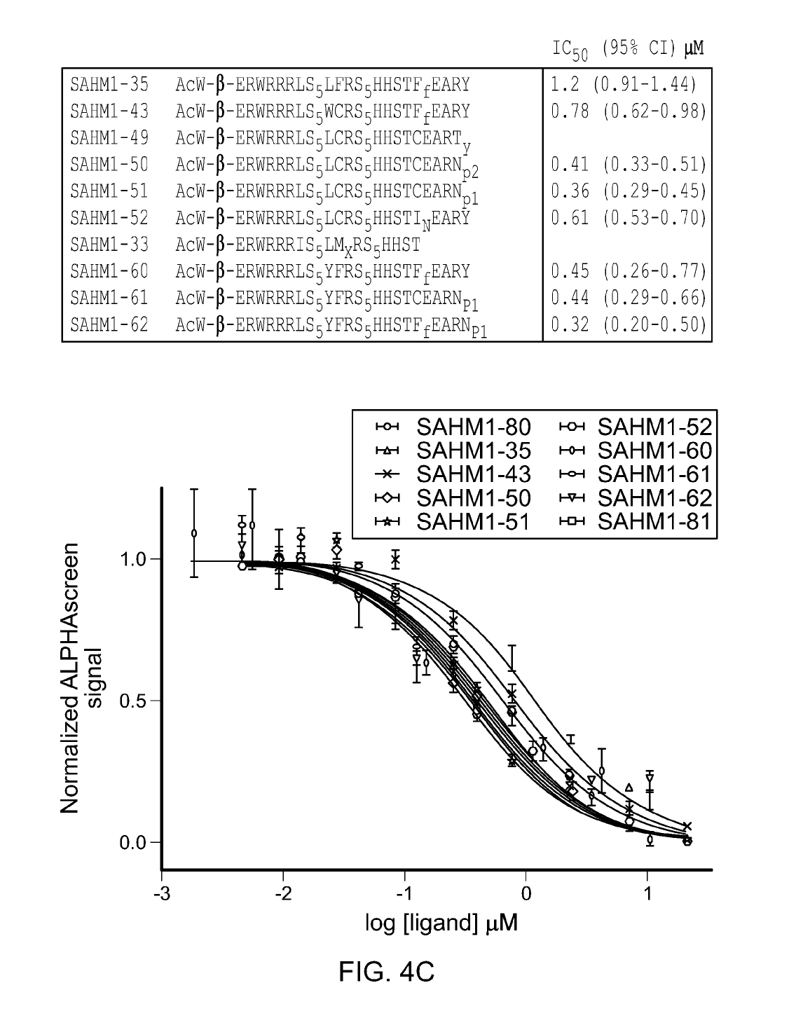

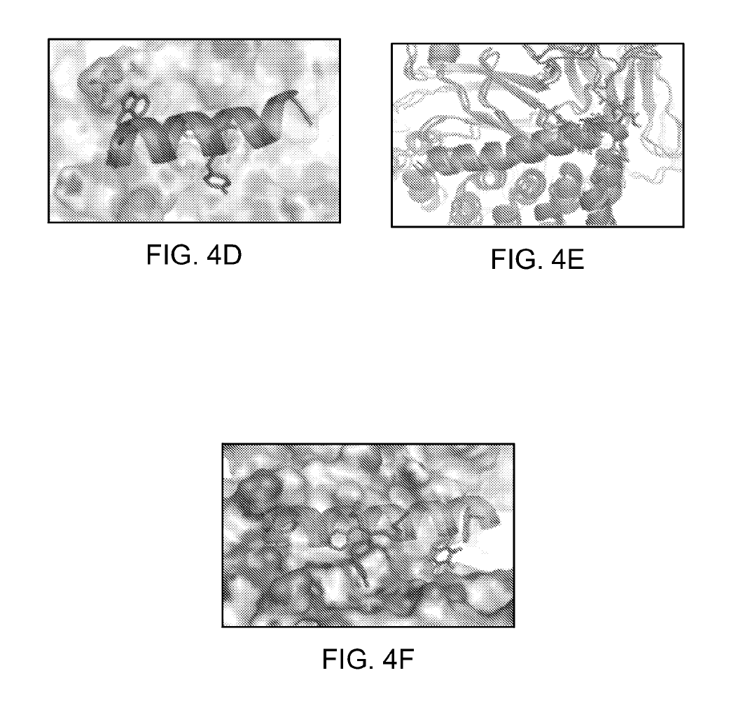

In addition to designing analog peptides derived from the E21-T36 region of dnMAML1, our BFED calculations (FIG. 2b,c) indicated that C-terminal extension of the SAHM1 scaffold might generate more potent and specific stapled peptides. Specifically, extension to Y4l (SAHM1-24) was expected to add interactions from C37, E38, R40 and Y4l in dnMAML1 (FIG. 3h). Extension to L49 (SAHM1-75) would further add potential contacts from E42, V44, E47 and L49, capping the ANK1 domain (FIG. 3i). These extended scaffold peptides were included in further rounds of optimization with the SAHM1 scaffold by incorporating favorable point mutations into multiple positions simultaneously. Initial combination mutants focused on determining the effect of I27 and L29 mutations in combination with the most effective point mutants separately—L23W and C30F—and then together (FIG. 4a). ALPHAscreen competition experiments revealed that L23W/I27L double mutants retain a gain in activity observed for the single mutants, but were without any significant gain for the double mutant (SAHM1-21/SAHM1-23 compared to SAHM1-31/SAHM1-36). This is likely due to the fact that both residues target the same hydrophobic cleft on CSL, which perhaps will not tolerate larger combinations. The L23W/C30F mutant combination appeared to improve peptide potency in the presence of different combinations of I27L and L29Y mutations (SAHM1-21, SAHM1-31 and SAHM1-56; FIG. 4d).

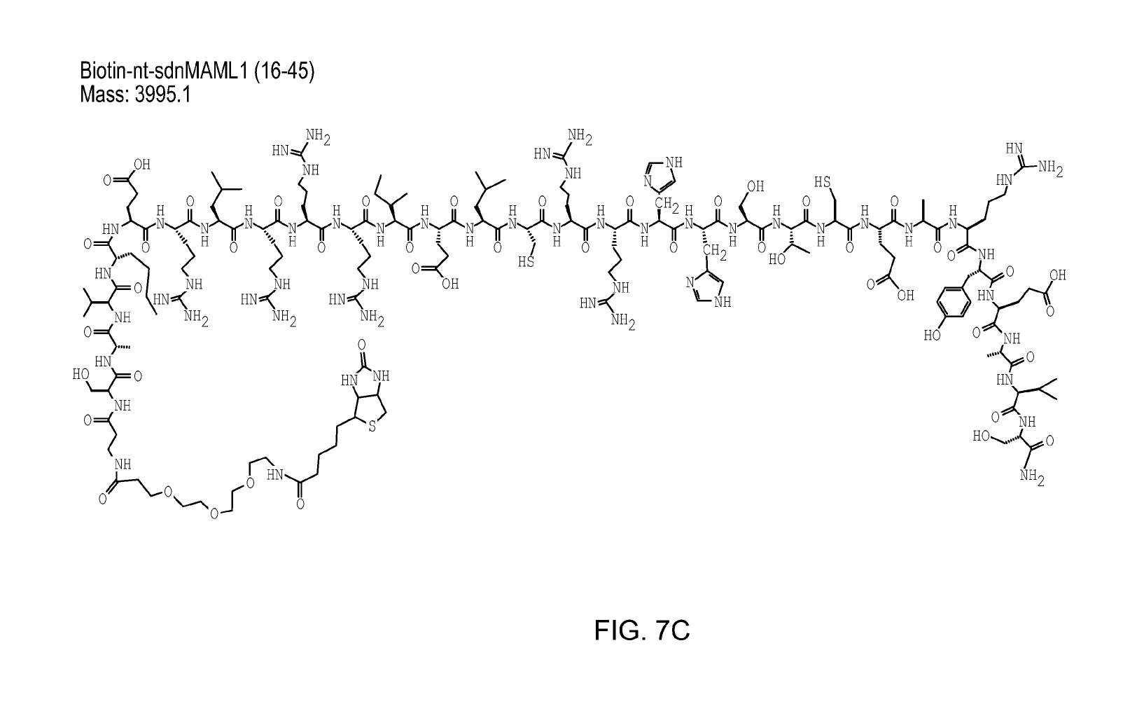

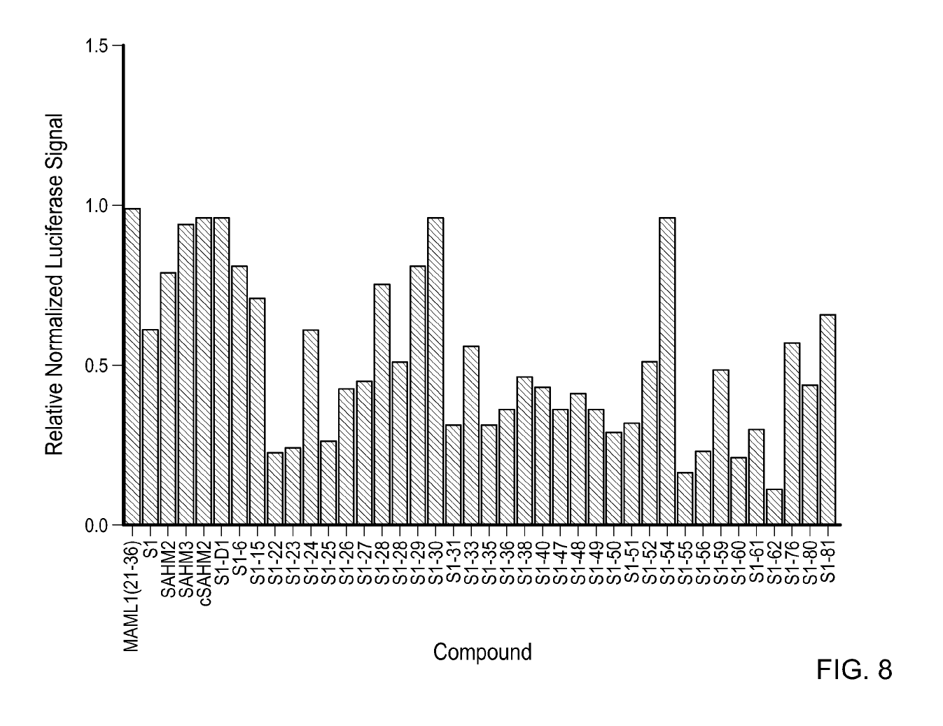

Additionally, the L23W/L29W combination mutations, which were separately found to improve competition and yield favorable MMGBSA scores, resulted in peptides with lower IC₅₀ values in combination with smaller substituents at C30 but not with the C30F mutant (FIG. 4a). These general SAR trends were also observed in the larger E21-Y4I stapled peptide scaffold, with more potent peptides containing the L23W/C30F and L23W/L29W double mutants (SAHM1-25, SAHM1-27, FIG. 4b). As peptides from the E21-Y41 scaffold are larger than those previously reported, decreased helicity might result in lower target affinity. To determine whether or not incorporation of multiple staples down the peptide backbone might improve activity, we synthesized two “stitched” peptides with staples spanning two sequential turns of the α-helix (SAHM1-29, SAHM1-30, FIG. 8). Interestingly, we found that neither was more active than their stapled peptide counterpart (SAHM1-24, FIG. 4b). Analogues from the largest peptide scaffold (E21-L49) were found to be more potent than SAHM1 and SAHM1-24 (FIG. 4b). Computational MD simulations of the most potent E21-L49 peptide, however, revealed stable binding for much of the peptide but with significant chaos in the C-terminal stretch after the proline-induced kink (FIG. 4e).

In an effort to take advantage of prospective structure-based design enabled by our NTC MD model, we were interested to determine if incorporation of non-natural amino acids could improve stapled peptide potency as well. In a similar approach to the aforementioned point mutation computational screen, we imported libraries of commercially available non-natural amino acids into our MD simulations. Non-natural amino acids were substituted within the E21-Y41 scaffold at promising sites for optimization as determined by MD, which included L23, C30, C37 and Y41. The resulting mutants were docked for each binding site-amino acid pair yielding MMGBSA free energy values and MD snapshots. Comparison of MMGBSA scores and docked structures suggested that peptides containing a handful of these non-natural amino acids could improve binding and the resulting peptides were synthesized (FIG. 4a, c; FIG. 8). From this effort several non-natural amino acids were found to retain relative peptide potency while introducing non-proteinogenic side chains (FIG. 4c). Notable examples were mutation of C37 to a D-pentafluoro phenylalanine and Y41 to 1-naphthylalanine (FIG. 4c,f). In contrast, some hits in our MD screen yielded peptides with significantly reduced activity, such as substitution of L23 with a nicotinyl-lysine amino acid (SAHM1-53, FIG. 4a). These results suggested that while our MD simulations can identify suitable non-proteinogenic residues for stapled peptide analogs, some predicted conformations might not be accessible and thus lead to deleterious interactions. In general, however, these SAR studies are in good agreement with the results of our initial MD simulations and have principally identified L23 and C30 as the most promising sites of optimization in both established (E21-T36) and novel (E21-Y41) stapled peptide scaffolds. MD snapshots of the relatively rigid hydrophobic cleft in CSL forming contacts with 127 revealed the potential for improvement, however the combination of mutants at 127 with the effective L23W mutation did show appreciable additive gains. Conversely, the relatively polar and flat surface on ANK1 targeted by L29 was the site of improvement through mutation to tyrosine or tryptophan in multiple combination mutant analogs. Inclusion of favorable mutations into stapled peptide scaffolds extended to Y41 and L49 was also found to yield increases in potency.

### Cell-Based Activity of Stapled Peptide Analogs

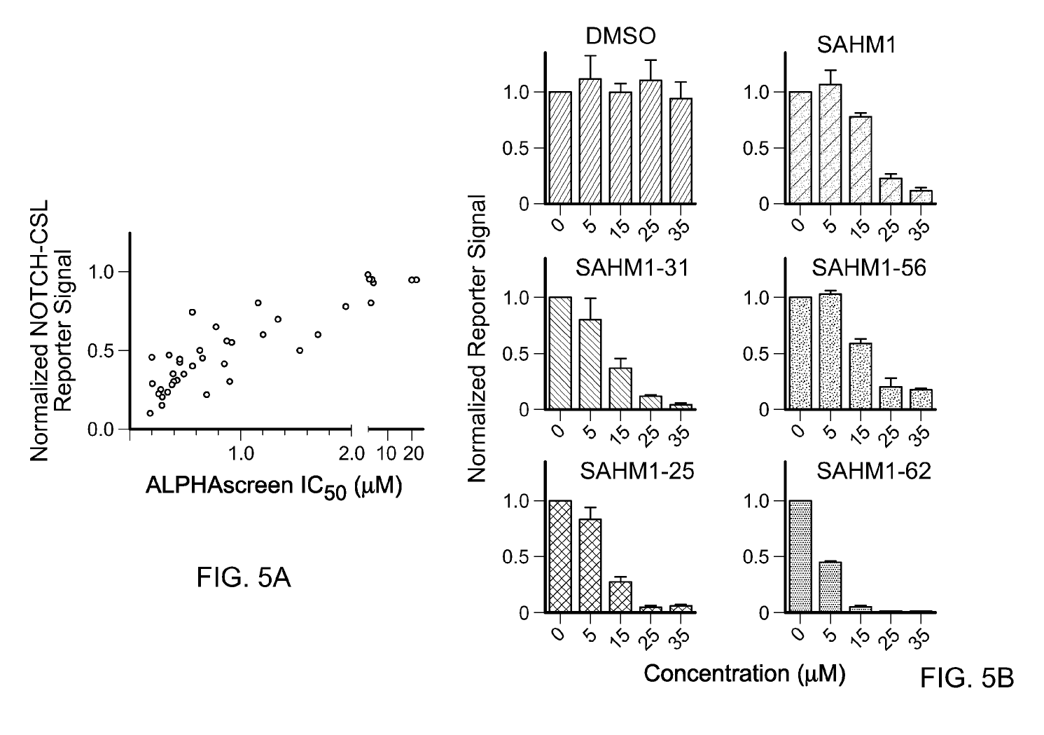

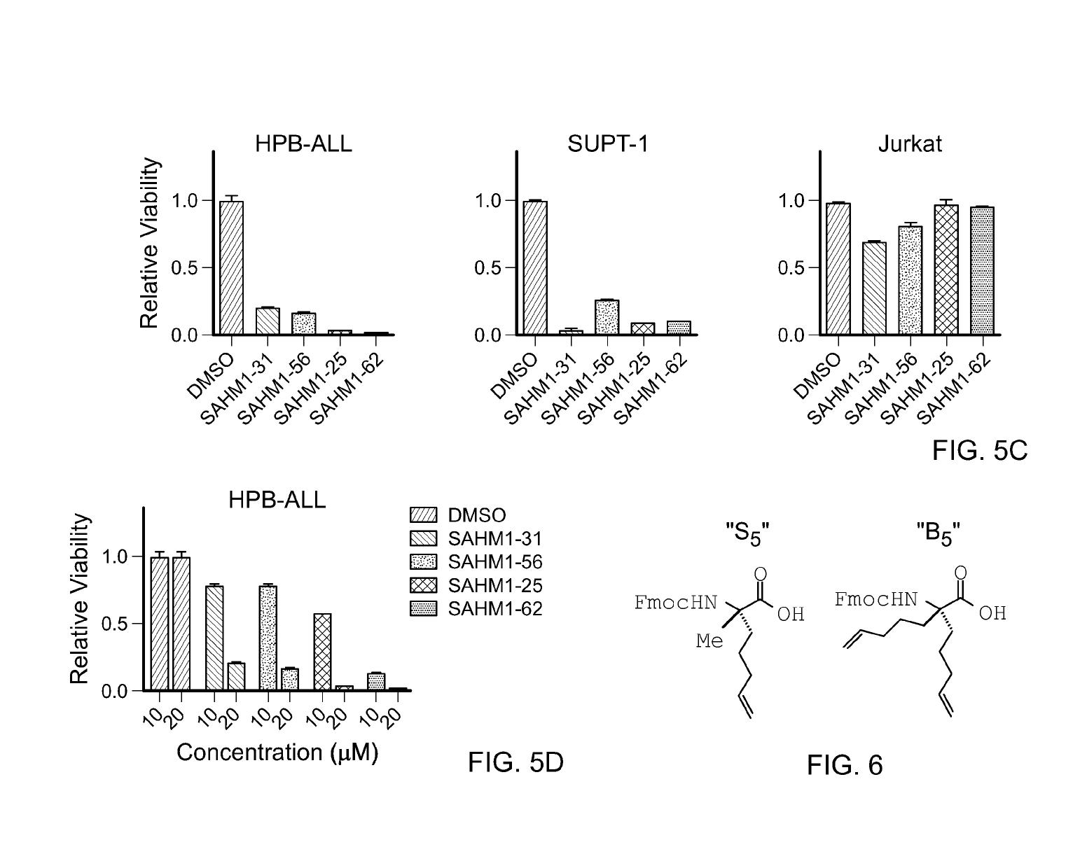

These SAR studies have established that analog stapled peptides based on three MAML1 scaffolds are capable of inhibiting NTC formation more potently than peptides based on wild type sequences alone. Despite the gains in activity observed, these improvements are not necessarily indicative of improved functional efficacy. In addition to target engagement, major factors governing cellular activity of stapled peptides are intracellular access, sub-cellular distribution and chemical stability. To determine whether the analogs described here were capable of antagonizing NOTCH1-CSL transactivation in cells, we tested all analogs in an established reporter-gene assay driven by constitutively activated NOTCH1^{37,46}. U2OS cells were co-transfected with a CSL-regulated firefly luciferase construct, a control Renilla-luciferase construct and the truncated ΔEGFΔLNR-NOTCH1 allele prior to treatment with analog compounds or vehicle. Comparison of stapled peptide IC₅₀ values in the ALPHAscreen assay and normalized inhibition of the NOTCH1-driven reporter gene signal revealed a strong correlation between biochemical and cell-based activity for the library of analogs (FIG. 5a, FIG. 9). This analysis indicated that more potent analogs from both the E21-T36 and E21-Y41 scaffolds were capable of nearly complete reporter repression (FIG. 5b). Interestingly, dose-dependent studies with optimized analogs from the shorter scaffold (SAHM1-31, SAHM1-56) revealed only slightly lower EC₅₀ values compared to AcW-SAHM1. Analogs based on the longer E21-Y41 scaffold (SAHM1-25, SAHM1-62) showed significantly lower EC₅₀ values of approximately 10 μM and 5 μM, respectively.

Notably, while peptides from the longest scaffold class (E21-L49) exhibited similar ALPHAscreen IC₅₀ values to SAHM1-25 and SAHM1-62, they caused only moderate repression of the NOTCH 1/CSL reporter signal (FIG. 5a).

Numerous studies have established that Notch pathway inhibition with γ-secretase inhibitors, monoclonal antibodies and stapled peptides leads to growth suppression and apoptosis in many human T-ALL cell lines that harbor activating NOTCH1 mutations^{46,5232,37}. Consistent with this, we found that treatment of two established NOTCH 1-dependent T-ALL cell lines, SUPT1 and HPB-ALL, with optimized SAHM analogs resulted in significantly decreased cell viability after three days (FIG. 5c). In contrast to HPB-ALL and SUPT1 cells, Jurkat T-ALL cells exhibit decreased sensitivity to Notch inhibitors owing to increased reliance on alternate signaling pathways^{53-55,37}. Treatment of Jurkat cells with the panel of analog SAHM peptides resulted in modest effects on cell proliferation after three days (FIG. 5c). Additionally, dose-dependent treatment of HPB-ALL cells inhibited proliferation at effective concentrations similar to those observed in the reporter assay for analog peptides (FIG. 5d). Together, these results confirm that optimized SAHM peptides from the E21-T36 and E21-Y41 scaffold classes inhibit NOTCH/CSL driven transcription and cell proliferation of NOTCH1-dependent T-ALL cell lines. They further indicate that optimized peptides from the E21-Y41 scaffold are more potent than peptides from the established E21-T36 and novel E21-L49 scaffolds.

Transcription factors represent some of the most attractive and validated targets in numerous diseases. Despite this, the discovery of synthetic modulators of this protein class has remained a challenging task for traditional drug discovery efforts. By incorporating the recognition properties of protein therapeutics with the synthetic accessibility of small molecules, hydrocarbon stapled peptides have demonstrated the capacity to target numerous intracellular protein-protein interactions with therapeutic potential. Reports detailing the design and characterization of novel stapled peptides to date have been primarily focused on the identification of peptides with the highest degree of structural stabilization and cell permeability^{41-43 37}. In all cases these two properties have been associated with the most active stapled peptides in cells and in vivo. Furthermore, these studies have primarily focused on stabilization of native peptide sequences with no attempt to alter and optimize binding interactions, which likely stems from the fact that high affinity short peptides had been described previously for the majority of these targets. In the present study we sought to use molecular modeling and structure-based design to quantitatively describe the protein-protein contacts involved in the assembly of the NTC and use these insights to design more potent stapled peptide inhibitors of the NOTCH complex. Predicting protein-protein binding conformations and interaction affinities has always been a challenging problem, since most of the binding surfaces are relatively large, flat and flexible. These characteristics make it difficult to apply scoring functions due to the need to search for much larger conformational space as well as predict binding affinities that are contributed to by numerous weak interactions. Here we employed molecular dynamics simulations of the NTC based on the human X-ray structure and performed binding free energy decomposition to calculate the extent to which each residue in dnMAML1, ANK1 and CSL contribute to complex formation and stability. The resulting model indicated that while dnMAML1 contact residues are distributed throughout the large helical interface, a majority of the contacts contributing strongly to complex formation are found in the N-terminal helix. These calculations agree with mutational studies⁴⁵ and our reported stapled peptide screen³⁷, suggesting that stabilized peptides derived from this region will have the highest ligand efficiency. We subsequently employed surface plasmon resonance to measure the binding affinity of a synthetic dnMAML1 peptide (s-dnMAML1) to a preformed RAMANK1-CSL complex. We found that s-dnMAML1, which contains all apparent contact residues in the human X-ray structure, bound the complex with high affinity (K_{D}=0.04 μM) and that truncation to the N-terminal helix (snt-dnMAML1) alone decreased the affinity by approximately 10-fold. These results support the contribution of both helices in high affinity complex binding via a “clamp-like” model as previously proposed⁵⁶, however the N-terminal helix alone does retain tight, specific binding. Together, these results represent the first reported binding affinities for dnMAML1 polypeptides to NOTCH-CSL complexes and provide a quantitative model of NTC complex formation. In addition to the application of this model for structure-based ligand optimization, as presented here, we posit that it could provide insights into the preferential formation of isoform-specific NTCs through quantitative analysis of isoform-specific residues in MAML1-3 and NOTCH1-4. Additionally, these models could be applied in future computational ligand discovery efforts, such as small molecule docking.

Using our MD model of the NTC we identified several hydrophobic residues in SAHM1 that if mutated might increase binding affinity. To calculate the relative effect of mutations at these positions, we also developed a stapled peptide MD simulation model that could be employed to compare unmodified native peptide sequences (MAML1 [21-36]), known stapled peptides (SAHM1), stapled peptide scaffolds containing mutations and novel stapled peptide scaffolds derived from MAML1. Simulation of the stapled peptide SAHM1 and its corresponding unmodified peptide revealed the significant stabilizing effect of the central hydrocarbon stapled in the peptide over the course of 20 ns, which is in agreement with previous circular dichroism experiments. This model was subsequently applied to calculate the relative BFED scores for a series of SAHM1 derivatives containing hydrophobic point-mutations as well as extended SAHM scaffolds derived from E21-Y41 and E21-L49 in MAML I. The results of these in silico screens supported the notion that mutation of residues L23 and C30, which both target hydrophobic surfaces on CSL, might improve binding. Additionally, MD simulations indicated that stapled peptides derived from E21-Y41 could improve NTC binding mainly through the addition of the Y41.

To determine whether the predictions of our MD model yielded compounds with improved activity, we developed a miniaturized, high-throughput ALPHAscreen competition assay that measures the association of a synthetic biotinylated dnMAML1 peptide with a GST-tagged RAMANK1-CSL protein complex. This assay proved to be specific to the presence of all complex members, displayed excellent signal-to-noise ratios and was sensitive to competition by unlabeled dnMAML1 or SAHM1 peptides. Therefore, we employed this assay to quantitatively profile SAHM analogs for NTC antagonism. Beyond this specific application, this assay represents a novel format to measure NOTCH complex formation and should be suitable for high-throughput screening of small molecules or other chemical classes.

In general, the results of our competitive ALPHAscreen assay profiling were in agreement with our MD simulations. Analogs containing the L23 W, C30F, I27L and L29W point-mutations showed the greatest inhibition of NTC formation at these respective positions. Subsequent incorporation of these mutations in relevant combinations within the SAHM1 and E21-Y41 scaffolds yielded peptides with IC₅₀ value improvements ranging from approximately three- to seven-fold. In both scaffolds the L23 W/C30F, L23 W/L29W, and L23 W/C30F/L29Y mutation combinations resulted in consistent ALPHAscreen IC₅₀ improvement. Peptides derived from the largest E21-L49 scaffold were also found to display improved biochemical activity relative to SAHM1 and SAHM1-24. Finally, taking advantage or the flexible design of stapled peptide analogs in our MD model, we sought to determine whether incorporation of non-natural amino acids into multiple positions might preserve or improve activity. By screening a commercially available library we identified a handful of promising non-proteinogenic mutations and synthesized the corresponding analog peptides. Notably, some of these residues were found to preserve activity, while others were found to significantly decrease activity. These results indicated that our model was capable of identifying productive interactions in some cases while the proposed binding conformations may not be accessible in others. As our search for non-natural residues was limited to a relatively small commercially available library, perhaps sampling a library with expanded chemical diversity in the future might yield more productive interactions.

The cell-based activity of reported stapled peptides in the literature has proven to be highly related to the degree of helical stabilization and cell penetration, rather than binding affinity alone. This phenomenon is attributed to the observation that stapled peptides enter cells through an active endocytotic mechanism, in contrast to most small molecules that enter through passive diffusion. With this in mind, a major goal of the work herein was to determine whether SAHM peptide biochemical potency could be optimized and to then determine the degree to which the cell-based activity was affected. All analog peptides were screened for inhibition of Notch signaling using an established NOTCH 1 reporter gene assay. Comparison of the resulting normalized reporter inhibition to the ALPHAscreen IC₅₀ for each peptide illustrated a positive correlation between the biochemical and cell-based activity for the library. Dose-dependent studies with four representative analog peptides revealed increased cell-based activity for all peptides relative to SAHM1, however the degree of IC₅₀ improvement was more modest than in the ALPHAscreen assay. The two most promising peptides, SAHM1-25 and SAHM1-62, exhibited IC₅₀ values that were approximately two-fold and four-fold lower than SAHM1, respectively. The two analogs from the E21-T36 scaffold showed improvements of 1.5- to 2-fold. In addition, analog peptides were found to have anti-proliferative effects on NOTCH 1-dependent T-ALL cell lines at equivalent effective concentrations as in the reporter gene assays. Taken together, these results support the notion that several stapled peptide analogs developed in this study are more potent inhibitors of Notch signaling. Additionally, our study suggests that while improved biochemical potency can contribute to increased cell-based activity, the phenomenon of active cellular uptake of stapled peptides might impose limitations to these improvements for stapled peptide inhibitors of the NTC.

### Experimental Procedures

Stapled Peptide Molecular Modeling—

The initial structure of stapled peptide SAHM1 was obtained based on the E21-T36 dnMAML1 in the human NOTCH complex³⁹ (PDBid: 2F8X) by mutating E28 and R32 to ligated α,α-disubstituted “S₅” amino acids. Conformational search of “S₅” non-natural amino acids were performed in Macromodel to generate the lowest energy conformation of SAHM1, which was then used as the starting coordinate for energy minimization, equilibration and 20 ns molecular dynamics simulation. The parameters of partial charge calculations, force fields for non-natural amino acids and MD simulations settings were described as follows.

NOTCH Complex Modeling—

The X-ray crystal structure of dnMAML1-ANK1-CSL bound to an oligo containing the HES1 promoter sequence (PDBid: 2f8x, 3.25 Å) was used as the starting coordinates for NTC MD simulations. The initial structure was processed in Protein Preparation Panel in Maestro 8.5. DNA and solvent molecules were removed from the structure. Protonation states were assigned to His, Gln, Asn residues and were manually inspected. The structure was then prepared in antechamber suite in Amber 10. In LEaP module, the ff03 force field in Amber 10 was used to simulate the system. Na⁺ was added to neutralize the system, which was then solvated in a TIP3P water box extending 10 Å from the complex. The final system contained around 700 amino acid residues. Protein minimization, equilibration and molecular dynamics simulations were carried out using SANDER.MPI module in Amber 10. Langevin dynamics was applied to control the temperature at 300 K while Particle-Mesh-Ewald (PME) summation was employed to treat long-range interactions. The SHAKE algorithm was used to allow an integration time step of 2 fs. 35 ns MD simulations were performed to study the flexible interactions between dnMAML1 and ANK1-CSL. Snapshots of the NTC were extracted every 10 ps from the last 30 ns of the MD simulation trajectories.

Computational Design of Non-Natural Amino Acids for MD Simulations—

For non-standard residues, we need to calculate the charges as well as the force field parameters for MD simulation. Since the non-standard residue is a central residue in the peptide, we add ACE and NME caps to it (CH₃CO—(NH—X—CO)—NHCH₃). Geometric optimization followed by single point charge calculation at HF/6-31 G* was applied in Gaussian 03. RESP program in antechamber suite was employed to fit the charges to atoms by restraining the total charge of the caps to zero. LEaP module in Amber 10 was used to prepare the structure. Non-natural residues were connected to the other residues and the missing parameters, like angles or dihedral, were carefully assigned using existing parameters from parm99, which describes atoms in very similar environment. Ff03 was used as the force field for the standard residues. Complexes with non-natural residues was then neutralized using Na+ and solvated by TIP3P water similar to the procedure described for NTC simulations.

dnMAML1 BFED Calculations—

Binding free energy decomposition (BFED) calculations are based on the average MMGBSA score of the ensemble of snapshots extracted every 10 ps from the converged 5-35 ns MD simulation of dnMAML1. BFED calculations are carried out using MMGBSA in Amber 10. Molecular mechanics method (MM) was applied to calculate the gas phase interaction energies between dnMAML1 and ANK1-CSL. The electrostatics component of solvation energy was calculated using Generalized Born (GB) method, while the non-polar solvation energy was estimated from the Solvent Accessible Surface Area (SASA). The entropy term was not included in our calculation, which is neither accurate nor necessary to compare peptide analogs that similar simplifications have been used by other researchers. BFED evaluates the contribution of each residue from two components (dnMAML or CSL/ANK) to the total binding free energy. So one half of the pairwise interaction energies, for example electrostatic interactions, are assigned to each of the two interacting atoms belonging to two residues respectively. The nonpolar contributions of each residue to the free energy of binding are proportional to the difference of the accessible surface of each residue in the free molecule and the complex.

SAHM Analogue Binding Calculations—

The starting structures for the MD simulations of SAHM analog complexes were obtained based on NOTCH complex X-ray structure (PDBid: 2F8X) by mutating respective residues of dnMAML. The methods to explore the lowest energy conformations of the mutated peptides in the complex, calculate partial charges and set up energy minimization, equilibration and MD simulations are very similar as described above. 18 ns MD simulations were applied for each of the SAHM analog complex. MMGBSA binding free energy calculations were performed based on the converged MD trajectories. MD trajectories were also analyzed to understand the dynamic behavior of the complex and explain how mutations affect the binding affinities.

Protein Expression and Purification—

Human CSL bearing a C-terminal hexahistidine tag (residues 9-435), RAMANK1 (residues 1761-2127) and GST-labeled RAMANK1 were expressed in BL21(DE3) pLysS cells (Stratagene) and purified as previously described³⁷.

Peptide Synthesis and Purification—

Stapled peptides were synthesized on a Tetras multi-channel automated peptide synthesizer (Thuramed) by standard Fmoc-based solid-phase peptide synthesis (SPPS) methods. Olefin-containing “S₅” and “B₅” amino acids and non-natural amino acids were purchased from Anaspec Inc. Following synthesis, ring-closing metathesis was performed using Grubbs I catalyst (benzylidene-bis(tricyclohexylphosphine)dichlororuthenium) in dichloroethane under nitrogen. All stapled peptides were subsequently capped with a beta-alanine spacer and an N-acetyl tryptophan to allow peptide quantification by absorbance at 280 nm. The theoretical extinction coefficients of 5500 M⁻¹cm⁻¹ and 1490 M⁻¹cm⁻¹ were used for tryptophan and tyrosine, respectively. Notably, previously reported peptides containing an N-terminal FITC label were not suitable for these studies due to spectral interference in the ALPHAscreen assay. Following synthesis, stapled peptides were cleaved from the resin, purified by reverse-phase HPLC on C18 column, quantified, lyophilized, resuspended in DMSO (5 to 10 mM) and stored at −20° C. Compound identification and purity was assessed by coupled liquid-chromatography mass-spectrometry (LCMS).

Synthetic dnMAML1 polypeptides were synthesized by SPPS using low-loading NovaPEG resin (EMD) on a CEM Liberty Microwave peptide synthesizer. Extended coupling time or double-coupling was used for beta-branched amino acids, stretches of hydrophobic residues and arginines. All couplings were performed at 70° C. with the exception of histidine and cysteine, which were coupled at 50° C. to prevent racemization. Biotinylated peptides (bio-s-dnMAML1 and bio-snt-dnMAML1) were capped with a beta-alanine spacer, a 20-atom diethylene glycol (EMD) spacer and biotin. Non-labeled competitor peptides (Ac-s-dnMAML1) were capped with an acetylated beta-alanine spacer. All peptides were cleaved, purified and quantified in the same manner as the stapled peptides.

ALPHAscreen Competition Assays—

Briefly, ALPHAscreen assays were performed using Perkin Elmer 384-well optiplates and measurements were made on a Perkin Elmer Envision multi-label plate reader with ALPHAscreen capability. Purified GST-RAMANK1 and CSL were dialyzed into binding buffer (20 mM Tris pH 8.4, 150 mM NaCl, 1 mM DTT, 1 mM EDTA and 0.05% dialyzed BSA) and kept separate for experiments. Briefly, 15 μL of 4× (of desired top concentration) stapled peptide stocks in binding buffer were added to the top row of plates containing 10 μL of binding buffer in all other wells. Serial three-fold dilutions were made leaving 10 μL in all wells. Notably, only non-fluorescent peptides were used in these assays as preliminary experiments indicated that the fluorophore interferes with the ALPHAscreen signal at mid-nanomolar concentrations. A 2× stock of CSL (80 nM), GST-RAMANK1 (80 nM) and Bio-sdnMAML1 (80 nM) was made in binding buffer and immediately added to wells containing diluted peptide stocks. This 30 μL solution was allowed to incubate at room temperature for 30 minutes. Separately. an 8× stock of anti-GST acceptor beads (160 μg/mL for a final concentration of 20 μg/mL) was resuspended in binding buffer in the dark. 5 μL of the acceptor bead solution was added to the wells, the plate centrifuged for 1 minute at 500 rpm and incubated for an addition 30 minutes. Finally, an 8× stock of streptavidin donor beads (160 μg/mL for a final concentration of 20 μg/mL) was made in binding buffer in the dark and 5 μL of the donor bead solution was added directly into the buffer (no centrifugation) and the 40 μL mixture was incubated for an addition 30 minutes at room temperature in the dark. The plate was read using standard ALPHAscreen settings and data processed using Prizm 5 software by applying non-linear regression analysis and fitting the data to a three-parameter sigmoidal curve.

Surface Plasmon Resonance—

A Biacore 3000 SPR-Instrument (Biacore-GE, Upsala, Sweden) was used to measure binding of Bio-sdnMAML1, Bio-sntdnMAML1 and Bio-SAHM1 peptides to soluble complexes of RAMANK1 and CSL. Peptides were dissolved in biacore binding buffer (20 mM Tris pH 8.4, 150 mM NaCl, 1 mM DTT, 1 mM EDTA and 0.05% P-20) and immobilized on a discrete flow cells of a streptavidin-CM5 Biacore chip by injection at 10 μL/min for 10 minutes. Equimolar dilutions of RAMANK1 and CSL (two-fold dilutions from 1 μM to 0.03125 μM, including two blanks) were mixed in biacore binding buffer and injected for 120-180 seconds onto the peptide-functionalized surface to measure NTC association kinetics, after which time NTC flow was stopped and buffer was injected to measure peptide dissociation kinetics. After the appropriate dissociation time (generally >6 minutes) the chip surface was regenerated using a high salt regeneration buffer (500 mM NaCl, 20 mM Tris pH 8.4 1 mM DTT and 0.05% P-20) to remove all bound complexes and prevent experimental carry-over. Binding data was reference-cell normalized and processed using ClampXP software: (http://www.cores.utah.edu/interaction/clamp.html). A two-site binding model was applied to the processed dataset to determine kinetic parameters of the peptide-NTC interactions.

Luciferase Reporter Gene Assays—

U2OS cells were plated in white, 96-well plates (Corning) containing DMEM supplemented with 10% FBS and allowed to acclimate overnight. Empty pcDNA3 or ΔEGFΔLNR-NOTCH1 plasmids (5 ng/well) were transiently co-transfected with a CSL-regulated firefly luciferase reporter construct and a constitutively active Renilla luciferase (pRLTK) control plasmid (10:1 Renilla:Firefly plasmid ratios) using Lipofectamine 3000 (Invitrogen)^{46 37}. Approximately 24-hours post-transfection cells were treated with DMSO vehicle control or stapled peptides at the given concentrations in fresh DMEM supplemented with 10% FBS and incubated for 18-24 hours. Luciferase activity was subsequently measured using a dual-luciferase assay kit (Promega) and NOTCH-dependent antagonism was measured by normalization of firefly and Renilla luciferase signals.

Cell Proliferation and Apoptosis Assays—

5×10⁴ cells were seeded in white, 96-well Corning plates in a total volume of 125 μL RPMI-1640 media containing 1% penicillin/streptomycin, 10% FBS and the indicated concentrations of DMSO or SAHM analog peptide. Cell viability was determined after three days by measuring cellular ATP content using the Cell Titer-Glo assay (Promega).

The structure, name, abbreviation and site(s) of introduction for non-natural amino acids used in analog stapled peptides is shown below.

| | Amino Acid | Mutant Site | Abbreviatio |
|---|---|---|---|
| 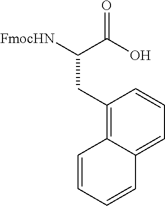 | Fmoc-(1-naphthyl)-L- Alanine-OH | Y41 | 1Np |
| 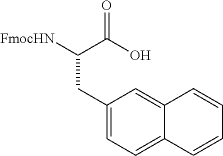 | Fmoc-(2-naphthyl)-L- Alanine-OH | Y41 | 2Np |
| 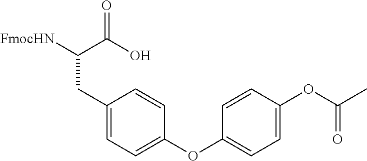 | Fmoc-(Ac)-L-Thyronine-OH | Y41 | Thy |
| 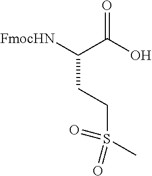 | Fmoc-L-Met(O₂)-OH | C30 | Mx |
| 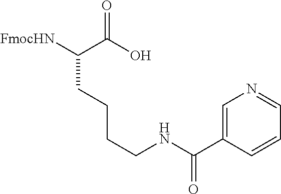 | Fmoc-L-Lys(Nicotinyl)-OH | L23 | Nk |
| 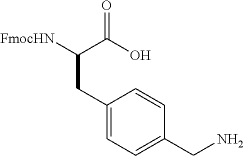 | Fmoc-D-4-methylamino)-Phe-OH | C37 | AmF |
| 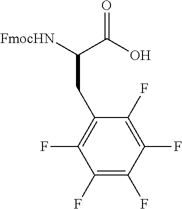 | Fmoc-D-pentafluoro-Phe-OH | C37 | pfF |

### Polypeptides

In some instances, the hydrocarbon cross-links described herein can be further manipulated. In one instance, a double bond of a hydrocarbon alkenyl cross-link, (e.g., as synthesized using a ruthenium-catalyzed ring closing metathesis (RCM)) can be oxidized (e.g., via epoxidation or dihydroxylation) to provide one of compounds below.

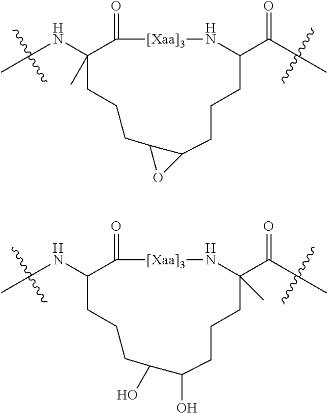

Either the epoxide moiety or one of the free hydroxyl moieties can be further functionalized. For example, the epoxide can be treated with a nucleophile, which provides additional functionality that can be used, for example, to attach a tag (e.g., a radioisotope or fluorescent tag). The tag can be used to help direct the compound to a desired location in the body or track the location of the compound in the body. Alternatively, an additional therapeutic agent can be chemically attached to the functionalized cross-link (e.g., an anti-cancer agent such as rapamycin, vinblastine, taxol, etc.). Such derivitization can alternatively be achieved by synthetic manipulation of the amino or carboxy terminus of the polypeptide or via the amino acid side chain. Other agents can be attached to the functionalized cross-link, e.g., an agent that facilitates entry of the polypeptide into cells.

While hydrocarbon cross-links have been described, other cross-links are also envisioned. For example, the cross-link can include one or more of an ether, thioether, ester, amine, 1,4-triazole, 1,5-triazole, hydrazone or amide moiety. In some cases, a naturally occurring amino acid side chain can be incorporated into the cross-link. For example, a cross-link can be coupled with a functional group such as the hydroxyl in serine, the thiol in cysteine, the primary amine in lysine, the acid in aspartate or glutamate, or the amide in asparagine or glutamine—all with or without inclusion of internal crosslinking moieties (such as biselectrophile-containing alkanes with a pair of cysteines, for example). Accordingly, it is possible to create a cross-link using naturally occurring amino acids rather than using a cross-link that is made by coupling two non-naturally occurring amino acids. It is also possible to use a single non-naturally occurring amino acid together with a naturally occurring amino acid.

It is further envisioned that the length of the cross-link can be varied: For instance, a shorter length of cross-link can be used where it is desirable to provide a relatively high degree of constraint on the secondary alpha-helical structure, whereas, in some instances, it is desirable to provide less constraint on the secondary alpha-helical structure, and thus a longer cross-link may be desired.

Additionally, while examples of cross-links spanning from amino acids i to i+3, i to i+4; and i to i+7 have been described in order to provide a cross-link that is primarily on a single face of the alpha helix, the cross-links can be synthesized to span any combinations of numbers of amino acids.

In some instances, alpha disubstituted amino acids are used in the polypeptide to improve the stability of the alpha helical secondary structure. However, alpha disubstituted amino acids are not required, and instances using mono-alpha substituents (e.g., in the cross-linked amino acids) are also envisioned.

As can be appreciated by the skilled artisan, methods of synthesizing the compounds of the described herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH, protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods. The peptides can be made in a high-throughput, combinatorial fashion. e.g., using a high-throughput multiple channel combinatorial synthesizer available from Advanced Chemtech. Long or complex peptides may also be made using microwave-assisted peptide synthesis, where standard solid-phase peptide synthesis methods are used in a reaction chamber enclosed in a controllable microwave apparatus. These methods permit rapid heating and cooling of the reaction environment, which can increase yields and access to otherwise difficult to synthesize peptides.

In the modified polypeptides one or more conventional peptide bonds replaced by a different bond that may increase the stability of the polypeptide in the body. Peptide bonds can be replaced by: a retro-inverso bonds (C(O)—NH); a reduced amide bond (NH—CH₂); a thiomethylene bond (S—CH₂ or CH₂—S); an oxomethylene bond (O—CH, or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. The polypeptides of the invention may also be conjugated to, for example, polyethylene glycol (PEG); alkyl groups (e.g., C1-C20 straight or branched alkyl groups); fatty acid radicals; and combinations thereof.

Methods of Treatment

The present invention provides for both prophylactic and therapeutic methods of treating a subject at risk of (or susceptible to) a disorder or having a disorder associated with aberrant (e.g., excessive) Notch activity or aberrant activity of a gene, gene-product or molecular signaling pathway that is regulated (positively or negatively) by Notch proteins (isoforms 1-4), MAML proteins (isoforms 1-3) and/or CSL. This is because the polypeptides are expected to act as dominant negative inhibitors of Notch-family, MAML-family and CSL protein activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The polypeptides of the invention can be used to treat, prevent, and/or diagnose cancers and neoplastic conditions. As used herein, the terms “cancer”, “hyperproliferative” and “neoplastic” refer to cells having the capacity for autonomous growth, i.e., an abnonnal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transfonned cells, tissues, or 30 organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Examples of cellular proliferative and/or differentiative disorders include cancer, e.g., carcinoma, sarcoma, or metastatic disorders. The compounds (i.e., the stapled polypeptides) can act as novel therapeutic agents for controlling breast cancer, T cell cancers and B cell cancer. The polypeptides may also be useful for treating mucoepidermoid carcinoma and medulloblastoma. Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Exemplary disorders include: acute leukemias, e.g., erythroblastic leukemia and acute mcgakaryoblastic leukemia. Additional exemplary myeloid disorders include, but are not limited to, acute promyeloid leukemia (APML), acute myelogenous 15 leukemia (AML) and chronic myelogenous leukemia (CML) (reviewed in Vaickus, L. (1991) Crit Rev. in Oncol. Hemotol. 11:267-97); lymphoid malignancies include, but are not limited to acute lymphoblastic leukemia (ALL) which includes B lineageALL and T-lineage ALL, chronic lymphocytic leukemia (eLL), prolymphocytic leukemia (PLL), multiple mylenoma, hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cellieukemiallymphoma (ATL), cutaneous T-cell lymphoma (CTCL), large granular lymphocytic leukemia (LGF), Hodgkin's disease and Reed-Sternberg disease. Examples of cellular proliferative and/or differentiative disorders of the breast include, but are not limited to, proliferative breast disease including, e.g, epithelial hyperplasia, sclerosing adenosis, and small duct papillomas; tumors, e.g., stromal tumors such as fibroadenoma, phyllodes tumor, and sarcomas, and epithelialtumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma. Other proliferative disorders that could be treated include cancers or metastatic disseminated tumors of the lung, pancreas, ovaries, gastrointestinal tract, liver as well as melanoma and medulloblastoma. The polypeptides could also be used for the treatment of any metastatic tumor on the basis of Notch-required signaling for angiogenesis (maintenance of blood supply) and cancer stem-cell like properties of metastatic cells. Cancers associated with hyperactivity of MAML-interacting proteins other than Notch and CSL, which include NF-kappa-B.

The polypeptides describe herein could be used for the treatment of many non-cancerous diseases associated with overactive Notch signaling, including osteoporosis, autoimmune disorders, inflammatory atherosclerosis and pulmonary hypertension. Additionally, other diseases associated with NF-kappa-B signaling, such as immunologic disorders, may be treated with the polypeptides herein. Furthermore, the polypeptides herein could be used for the treatment (in vivo or ex vivo) of tissues or cells from patients for regenerative medicine or stem cell therapy.

### Pharmaceutical Compositions and Routes of Administration

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate. nitrate, palmoate, phosphate, picrate, pivalate, propionate, salicylate, succinate, sulfate, tartrate, tosylate, trifluoromethylsulfonate, and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

The compounds of the formulae described herein can, for example, be administered by injection, intravenously, intraarterially, subdermally, intraperitoneally, intramuscularly, or subcutaneously; or orally, buccally, nasally, transmucosally. topically, in an ophthalmic preparation, or by inhalation, with a dosage ranging from about 0.001 to about 100 mg/kg of body weight, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary.

Subsequently, the dosage or frequency of administration, or both, may be reduced, as a function of the symptoms, to a level at which the improved condition is retained. Patients may, however, require intermittent treatment on a long-term basis upon any recurrence of disease symptoms.

Pharmaceutical compositions of this invention comprise a compound of the formulae described herein or a pharmaceutically acceptable salt thereof; an additional agent including for example, morphine or codeine; and any pharmaceutically acceptable carrier, adjuvant or vehicle. Alternate compositions of this invention comprise a compound of the formulae described herein or a pharmaceutically acceptable salt thereof; and a pharmaceutically acceptable carrier, adjuvant or vehicle. The compositions delineated herein include the compounds of the formulae delineated herein, as well as additional therapeutic agents if present, in amounts effective for achieving a modulation of disease or disease symptoms. The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

### Modification of Polypeptides

The stapled polypeptides can include a drug, a toxin, a derivative of polyethylene glycol; a second polypeptide; a carbohydrate, etc. Where a polymer or other agent is linked to the stapled polypeptide is can be desirable for the composition to be substantially homogeneous.

The addition of polyethelene glycol (PEG) molecules can improve the pharmacokinetic and pharmacodynamic properties of the polypeptide. For example, PEGylation can reduce renal clearance and can result in a more stable plasma concentration. PEG is a water soluble polymer and can be represented as linked to the polypeptide as formula:

XO—(CH₂CH₂O)ₙ—CH₂CH₂—

Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to an agent described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C1-C6) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

In some cases solubility and/or alpha helicity can sometime be improved by modifying the amino-terminus of the peptide to attach spermine (Muppidi et al. 2011 Bioorg Med. Chem Lett 7412).

### Screening Assays

The assays described herein can be performed with individual candidate compounds or can be performed with a plurality of candidate compounds. Where the assays are performed with a plurality of candidate compounds, the assays can be performed using mixtures of candidate compounds or can be run in parallel reactions with each reaction having a single candidate compound. The test compounds or agents can be obtained using any of the numerous approaches in combinatorial library methods known in the art.

### Other Applications

A number of embodiments of the invention have been described. Nevertheless, it will be understood that various modifications may be made without departing from the spirit and scope of the invention. Accordingly, other embodiments are within the scope of the following claims.

### REFERENCES

1. Darnell, J. E. J. Transcription factors as targets for cancer therapy. Nat Rev Cancer 2, 740-749 (2002).
2. Artavanis-Tsakonas, S., Rand, M. D. & Lake, R. J. Notch signaling: cell fate control and signal integration in development. Science 284, 770-776 (1999).
3. Bray, S. J. Notch signalling: a simple pathway becomes complex. Nat Rev Mol Cell Biol 7, 678-689 (2006).
4. Joutel, A. et al. Notch3 mutations in CADASIL, a hereditary adult-onset condition causing stroke and dementia. Nature 383, 707-710 (1996).
5. Garg, V. et al. Mutations in NOTCH1 cause aortic valve disease. Nature 437, 270-274 (2005).
6. Li, L. et al. Alagille syndrome is caused by mutations in human Jagged 1. which encodes a ligand for Notch1. Nat Genet 16, 243-251 (1997).
7. Ellisen, L. W. et al. TAN-1, the human homolog of the Drosophila notch gene, is broken by chromosomal translocations in T lymphoblastic neoplasms. Cell 66, 649-661 (1991).
8. Weng, A. P. et al. Activating mutations of NOTCH 1 in human T cell acute lymphoblastic leukemia. Science 306, 269-271 (2004).
9. Colaluca, I. N. et al. NUMB controls p53 tumour suppressor activity. Nature 451, 76-80 (2008).
10. Park, J. T. et al. Notch3 gene amplification in ovarian cancer. Cancer Res 66, 6312-6318 (2006).
11. Konishi, J. et al. Gamma-secretase inhibitor prevents Notch3 activation and reduces proliferation in human lung cancers. Cancer Res 67, 8051-8057 (2007).
12. Westhoff, B. et al. Alterations of the Notch pathway in lung cancer. Proc Natl Acad Sci USA 106, 22293-22298 (2009).
13. De La, O. J. P. et al. Notch and Kras reprogram pancreatic acinar cells to ductal intraepithelial neoplasia. Proc Natl Acad Sci USA 105, 18907-18912 (2008).
14. Pinnix, C. C. et al. Active Notch1 confers a transformed phenotype to primary human melanocytes. Cancer Res 69, 5312-5320 (2009).
15. Nefedova, Y., Cheng, P., Alsina, M., Dalton, W. S. & Gabrilovich, D. I. Involvement of Notch-1 signaling in bone marrow stroma-mediated de novo drug resistance of myeloma and other malignant lymphoid cell lines. Blood 103, 3503-3510 (2004).
16. Fung, E. et al. Delta-like 4 induces notch signaling in macrophages: implications for inflammation. Circulation 115, 2948-2956 (2007).
17. Niranjan, T. et al. The Notch pathway in podocytes plays a role in the development of glomerular disease. Nat Med 14, 290-298 (2008).
18. Hilton, M. J. et al. Notch signaling maintains bone marrow mesenchymal progenitors by suppressing osteoblast differentiation. Nat Med 14, 306-314 (2008).
19. Li, X. et al. Notch3 signaling promotes the development of pulmonary arterial hypertension. Nat Med 15, 1289-1297 (2009).
20. Brou, C. et al. A novel proteolytic cleavage involved in Notch signaling: the role of the disintegrin-metalloprotease TACE. Mol Cell 5, 207-216 (2000).
21. Struhl, G. & Greenwald, I. Presenilin is required for activity and nuclear access of Notch in Drosophila. Nature 398, 522-525 (1999).
22. Ye, Y., Lukinova, N. & Fortini, M. E. Neurogenic phenotypes and altered Notch processing in Drosophila Presenilin mutants. Nature 398, 525-529 (1999).
23. De Strooper, B. et al. A presenilin-1-dependent gamma-secretase-like protease mediates release of Notch intracellular domain. Nature 398, 518-522 (1999).
24. Wu, L. et al. MAML1, a human homologue of Drosophila mastermind, is a transcriptional co-activator for NOTCH receptors. Nat Genet 26, 484-489 (2000).
25. Fryer, C. J., Lamar, E., Turbachova, I., Kintner, C. & Jones, K. A. Mastermind mediates chromatin-specific transcription and turnover of the Notch enhancer complex. Genes Dev 16, 1397-1411 (2002).
26. Oswald, F. et al. RBP-Jkappa/SHARP recruits CtIP/CtBP corepressors to silence Notch target genes. Mol Cell Biol 25, 10379-10390 (2005).
27. Kovall, R. A. & Hendrickson, W. A. Crystal structure of the nuclear effector of Notch signaling, CSL, bound to DNA. EMBO J 23, 3441-3451 (2004).
28. Nam, Y., Weng, A. P., Aster, J. C. & Blacklow, S. C. Structural requirements for assembly of the CSL.intracellular Notch1.Mastermind-like 1 transcriptional activation complex. J Biol Chem 278, 21232-21239 (2003).
29. Noguera-Troise, 1. et al. Blockade of D114 inhibits tumour growth by promoting non-productive angiogenesis. Nature 444, 1032-1037 (2006).
30. Ridgway, J. et al. Inhibition of D114 signalling inhibits tumour growth by deregulating angiogenesis. Nature 444, 1083-1087 (2006).
31. Li, K. et al. Modulation of Notch signaling by antibodies specific for the extracellular negative regulatory region of NOTCH3. J Biol Chem 283, 8046-8054 (2008).
32. Wu, Y. et al. Therapeutic antibody targeting of individual Notch receptors. Nature 464, 1052-1057 (2010).
33. Tian, G. et al. Linear non-competitive inhibition of solubilized human gamma-secretase by pepstatin A methylester, L685458, sulfonamides, and benzodiazepines. J Biol Chem 277, 31499-31505 (2002).
34. Dovey, H. F. et al. Functional gamma-secretase inhibitors reduce beta-amyloid peptide levels in brain. J Neurochem 76, 173-181 (2001).
35. Seiffert, D. et al. Presenilin-1 and -2 are molecular targets for gamma-secretase inhibitors. J Biol Chem 275, 34086-34091 (2000).
36. Sparey, T. et al. Cyclic sulfamide gamma-secretase inhibitors. Bioorg Med Chem Lett 15, 4212-4216 (2005).
37. Moellering, R. E. et al. Direct inhibition of the NOTCH transcription factor complex. Nature 462, 182-188 (2009).
38. Wilson, J. J. & Kovall, R. A. Crystal structure of the CSL-Notch-Mastermind ternary complex bound to DNA. Cell 124, 985-996 (2006).
39. Nam, Y., Sliz, P., Song, L., Aster, J. C. & Blacklow, S. C. Structural basis for cooperativity in recruitment of MAML coactivators to Notch transcription complexes. Cell 124, 973-983 (2006).
40. Schafmeister, C. E., Po, J. & Verdine, G. L. An all-hydrocarbon cross-linking system for enhancing the helicity and metabolic stability of peptides. J Am Chem Soc 122, 5891-5892 (2000).
41. Walensky, L. D. et al. Activation of apoptosis in vivo by a hydrocarbon-stapled BH3 helix. Science 305, 1466-1470 (2004).
42. Bernal, F., Tyler, A. F., Korsmeyer, S. J., Walensky, L. D. & Verdine, G. L. Reactivation of the p53 tumor suppressor pathway by a stapled p53 peptide. J Am Chem Soc 129, 2456-2457 (2007).
43. Danial, N. N. et al. Dual role of proapoptotic BAD in insulin secretion and beta cell survival. Nat Med 14, 144-153 (2008).
44. Zhang, H. et al. A cell-penetrating helical peptide as a potential HIV-1 inhibitor. J Mol Biol 378, 565-580 (2008).
45. Del Bianco, C., Aster, J. C. & Blacklow, S. C. Mutational and energetic studies of Notch 1 transcription complexes. J Mol Biol 376, 131-140 (2008).
46. Weng, A. P. et al. Growth suppression of pre-T acute lymphoblastic leukemia cells by inhibition of notch signaling. Mol Cell Biol 23, 655-664 (2003).
47. Kutchukian, P. S., Yang, J. S., Verdine, G. L. & Shakhnovich, E. I. All-atom model for stabilization of alpha-helical structure in peptides by hydrocarbon staples. J Am Chem Soc 131, 4622-4627 (2009).
48. Guo, Z. et al. Probing the alpha-helical structural stability of stapled p53 peptides: molecular dynamics simulations and analysis. Chem Biol Drug Des 75, 348-359 (2010).
49. Lubman, 0. Y., Ilagan, M. X., Kopan, R. & Barrick, D. Quantitative dissection of the Notch:CSL interaction: insights into the Notch-mediated transcriptional switch. J Mol Biol 365, 577-589 (2007).
50. Ullman, E. F. et al. Luminescent oxygen channeling immunoassay: measurement of particle binding kinetics by chemiluminescence. Proc Natl Acad Sci USA 91, 5426-5430 (1994).
51. Eglen, R. M. et al. The use of AlphaScreen technology in HTS: current status. Curr Chem Genomics 1, 2-10 (2008).
52. Lewis, H. D. et al. Apoptosis in T cell acute lymphoblastic leukemia cells after cell cycle arrest induced by pharmacological inhibition of notch signaling. Chem Biol 14, 209-219 (2007).
53. O′Neil, J. et al. FBW7 mutations in leukemic cells mediate NOTCH pathway activation and resistance to gamma-secretase inhibitors. J Exp Med 204, 1813-1824 (2007).
54. Rao, S. S. et al. Inhibition of NOTCH signaling by gamma secretase inhibitor engages the RB pathway and elicits cell cycle exit in T-cell acute lymphoblastic leukemia cells. Cancer Res 69, 3060-3068 (2009).
55. Palomero, T. et al. Mutational loss of PTEN induces resistance to NOTCH1 inhibition in T-cell leukemia. Nat Med 13, 1203-1210 (2007).
56. Friedmann, D. R., Wilson, J. J. & Kovall, R. A. RAM-induced allostery facilitates assembly of a notch pathway active transcription complex. J Biol Chem 283, 14781-14791 (2008).

1. An internally cross-linked polypeptide comprising the amino acid sequence of any of SEQ ID NOs 12-20, wherein the side chains of at least two amino acids separated by three or six amino acids are replaced by an internal cross-link.

2. The internally cross-linked polypeptide of claim 1 wherein:
(a) the side chains of a first, a second and a third amino acid are replaced by internal cross-links;
(b) the first and second amino acids are separated by three or six amino acid and the second and third amino acids are separated by three or six amino acids; and
(c) there is an internal cross-link between the first and second amino acid and an internal cross-link between the second and third amino acids.

3. The internally cross-linked polypeptide of claim 1 wherein the side chains of Xaa8 and Xaa12 are replaced by an internal cross-link or the side chains of Xaa4 and Xaa8 are replaced by an internal cross-link or the side chains of Xaa12 and Xaa16 are replaced by an internal cross-link.

4. A modified polypeptide of Formula (I), 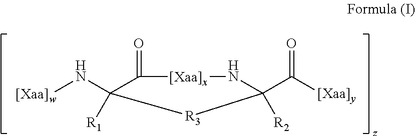
or a pharmaceutically acceptable salt thereof,
wherein:
each R₁ and R₂ are independently H, alkyl, alkenyl, alkynyl, arylalkyl, cycloalkylalkyl, heteroarylalkyl, or heterocyclylalkyl;
each R₃ is independently alkyl, alkenyl, alkynyl; [R₄-K-R₄′]ₙ; each of which is substituted with 0-6 R₅;
R₄ and R₄′ are independently alkylene, alkenylene or alkynylene;
each R₅ is independently is halo, alkyl, OR₆, N(R₆)₂, SR₆, SOR₆, SO₂R₆, CO₂R₆, R₆, a fluorescent moiety, or a radioisotope;
each K is independently O, S, SO, SO₂, CO, CO₂, CONR₆, or 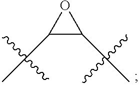 [SMILES: *C1OC1*]
each R₆ is independently H, alkyl, or a therapeutic agent;
n is an integer from 1-4;
x is 2, 3 or 6;
y and w are independently integers from 0-100;
z is an integer from 1-10; and
each Xaa is independently an amino acid;
wherein the modified polypeptide comprises at least 8 contiguous amino acids of any of SEQ ID NOs:12-20 except that: (a) within the 8 contiguous amino acids the side chains of at least one pair of amino acids separated by 3, 4 or 6 amino acids is replaced by the linking group R₃ which connects the alpha carbons of the pair of amino acids as depicted in Formula I and (b) the alpha carbon of the first amino acid of the pair of amino acids is substituted with R₁ as depicted in formula I and the alpha carbon of the second amino acid of the pair of amino acids is substituted with R₂ as depicted in Formula I.

5. The modified polypeptide of claim 4, wherein the modified polypeptide binds to ICN1-CSL.

6. The modified polypeptide of claim 4, wherein x is 2.

7. The modified polypeptide of claim 4, wherein x is 3.

8. The modified polypeptide of claim 4, wherein x is 6.

9. The modified polypeptide of claim 4, wherein x is 2, 3 or 6; R₃ is an alkenyl containing a single double bond, and both R₁ and independently R₂ are H or methyl.

10. The modified polypeptide of claim 4, wherein each y is independently an integer between 3 and 15.

11. The modified polypeptide of claim 4, wherein the polypeptide comprises at least 16 contiguous amino acids of any SEQ ID NO:12-20 except that: (a) within the 8 contiguous amino acids the side chains of at least one pair of amino acids separated by 3, 4 or 6 amino acids is replaced by the linking group R₃ which connects the alpha carbons of the pair of amino acids as depicted in Formula I and (b) the alpha carbon of the first amino acid of the pair of amino acids is substituted with R₁ as depicted in formula I and the alpha carbon of the second amino acid of the pair of amino acids is substituted with R₂ as depicted in Formula I.

12. The modified polypeptide of claim 4 comprising at least 16 contiguous amino acids of Glu₁Arg₂Xaa₃Xaa₄Arg₅Arg₆Xaa₇Xaa₈Xaa₉Xaa₁₀Arg₁₁Xaa₁₂HiS₁₃His₁₄ Ser₁₅Xaa₁₆ (SEQ ID NO:12)
wherein the side chains of Xaa₄ and Xaa₈ are replaced the linking group R₃ as depicted in Formula I which connects the alpha carbons of the pair of amino acids and the alpha carbon of the first amino acid of the pair of amino acids is substituted with R₁ as depicted in formula I and the alpha carbon of the second amino acid of the pair of amino acids is substituted with R₂ as depicted in Formula I.

13. The modified polypeptide of claim 4 wherein the polypeptide does not have a net negative charge at pH 7.

14. The modified polypeptide of claim 4 wherein the polypeptide comprises at least one amino acid that has a positive charge at pH 7.

15. The modified polypeptide of claim 4 wherein the polypeptide is covalently bound to PEG.

16. The modified polypeptide of claim 4, wherein R₁ and R₂ are each independently H or C₁-C₆ alkyl.

17-21. (canceled)

22. The modified polypeptide of claim 4, wherein x is 6.

23. The modified polypeptide of claim 22, wherein R₃ is C₁₁ alkenyl.

24. The modified polypeptide of claim 1, wherein R₃ is alkenyl.

25. A modified polypeptide of Formula (II), 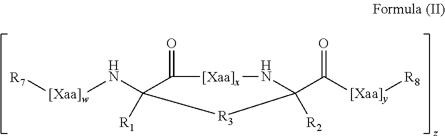
or a pharmaceutically acceptable salt thereof,
wherein;
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkylene, alkenylene or alkynylene, or [R₄′-K-R₄]ₙ; each of which is substituted with 0-6 R₅;
R₄ and R₄′ are independently alkylene, alkenylene or alkynylene (e.g., each are independently a C1, C2, C3, C4, C5, C6, C7, C8, C9 or C10 alkylene, alkenylene or alkynylene);
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or 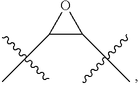 [SMILES: *C1OC1*]
aziridine, episulfide, diol, amino alcohol;
R₆ is H, alkyl, or a therapeutic agent;
n is 2, 3, 4 or 6;
x is an integer from 2-10;
w and y are independently an integer from 0-100;
z is an integer from 1-10 (e.g., 1, 2, 3, 4, 5, 6, 7, 8, 9, 10); and
each Xaa is independently an amino acid (e.g., one of the 20 naturally occurring amino acids or any naturally occurring non-naturally occurring amino acid);
R₇ is PEG, a tat protein, an affinity label, a targeting moiety, a fatty acid-derived acyl group, a biotin moiety, a fluorescent probe (e.g. fluorescein or rhodamine) linked via, e.g., a thiocarbamate or carbamate linkage;
R₈ is H, OH, NH₂, NHR₈ₐ, NR₈ₐR_{8b};
wherein the polypeptide comprises at least 8 contiguous amino acids of any of SEQ ID NOs:12-20 or another polypeptide sequence described herein except that: (a) within the 8 contiguous amino acids of any of SEQ ID NOs:12-20 wherein the side chains of at least one pair of amino acids separated by 3, 4 or 6 amino acids is replaced by the linking group, R₃, which connects the alpha carbons of the pair of amino acids as depicted in formula I; and (b) the alpha carbon of the first of the pair of amino acids is substituted with R₁ as depicted in Formula II and the alpha carbon of the second of the pair of amino acids is substituted with R₂ as depicted in Formula II.

26-28. (canceled)
